SEQUENCE LISTING (13 sequences)

SEQ ID NO: 1 - DNA (cDNA), 1708 bp; single-stranded, linear
  CDS 249..1001
gacgcttctgatctccagaggaccctggggtgggacaggggccttggcaaggctgcagccgctgggcagtggcttggaatggaggtctttattactgggaactgaggagctaagaggctcctgtcagcttgtcctaaagtcttagcacttgtggtggcttgggcttcacacactgtcagacaccttcgtggtggcctccacggcctcaccttcaggtttgaagctggctccacaagggacacggtgacatgaggggcagcccactgatccatcttctggccacttccttcctctgccttctctcaatggtgtgtgcccagctgtgccggacaccctgtacctgtccttggacaccaccccagtgcccacagggggtacccctggtgctggatggctgtggctgctgtaaagtgtgtgcacggaggctgggggagtcctgcgaccacctgcatgtctgcgaccccagccagggcctggtttgtcagcctggggcaggccctggcggccatggggctgtgtgtctcttggatgaggatgacggtagctgtgaggtgaatggccgcaggtacctggatggagagacctttaaacccaattgcagggtcctgtgccgctgtgatgacggtggcttcacctgcctgccgctgtgcagtgaggatgtgcggctgcccagctgggactgcccacgccccaagagaatacaggtgccaggaaagtgctgccccgagtgggtatgtgaccagggagtgacaccggcgatccagcgctccacggcgcaaggacaccaactttctgcccttgtcactcctgcctctgctgatgctccttgtccaaattggagcacagcctggggcccctgctcaaccacctgtgggctgggcatagccacccgagtgtccaaccagaaccgattctgccaactggagatccaacgccgcctgtgtctgcccagaccctgcctggcagccaggagccacagctcatggaacagtgctttctaaggccaactggggatgcggatacagggcctgccatcctcagcaaatgaccctaggaccaggccctggactgctggtagatgctcttctccatgctcttggctgcagttaactgtcctgcttggattcactgtgtagagccactgagcgatccctgctctgtctgaggtaggcggagcaggtgaccagctccagttctctggttcagcctggaattctgggttctcctggctcattcctcaaaacatccctgtacaaaaaggacaaccaaaaagacctttaaacctaggctatactgggcaaacctggccaccgtgctggggataaggtcaatgttaggaccagacagcagattgcctgaaacttccaattcccttcttggacttctgtatgcttgtccccaaagatgatgaatgaactcgtaagtgtaccttccctgacctgagaacaccctgcctgctcgggaagtattcaggggcagaattctctgtgaacatgaagagatgaatcacactgtccttaagaaattcctgaaagtccaggaacttgagctttgtattttcaggaatgcacatctcttaagcactcgcaaaacaggaaggctccacacctctggcaggccagggcctttctcttcagcatgagaaagacaagggacagcagagtactctcctctggaggactagtctagcctagaataaacacccaaa

SEQ ID NO: 2 - protein, 250 aa
MRGSPLIHLLATSFLCLLSMVCAQLCRTPCTCPWTPPQCPQGVPLVLDGCGCCKVCARRLGESCDHLHVCDPSQGLVCQPGAGPGGHGAVCLLDEDDGSCEVNGRRYLDGETFKPNCRVLCRCDDGGFTCLPLCSEDVRLPSWDCPRPKRIQVPGKCCPEWVCDQGVTPAIQRSTAQGHQLSALVTPASADAPCPNWSTAWGPCSTTCGLGIATRVSNQNRFCQLEIQRRLCLPRPCLAARSHSSWNSAF

SEQ ID NO: 3 - DNA (cDNA), 753 bp; single-stranded, linear
  CDS 1..750
atgaggggcagcccactgatccatcttctggccacttccttcctctgccttctctcaatggtgtgtgcccagctgtgccggacaccctgtacctgtccttggacaccaccccagtgcccacagggggtacccctggtgctggatggctgtggctgctgtaaagtgtgtgcacggaggctgggggagtcctgcgaccacctgcatgtctgcgaccccagccagggcctggtttgtcagcctggggcaggccctggcggccatggggctgtgtgtctcttggatgaggatgacggtagctgtgaggtgaatggccgcaggtacctggatggagagacctttaaacccaattgcagggtcctgtgccgctgtgatgacggtggcttcacctgcctgccgctgtgcagtgaggatgtgcggctgcccagctgggactgcccacgccccaagagaatacaggtgccaggaaagtgctgccccgagtgggtatgtgaccagggagtgacaccggcgatccagcgctccacggcgcaaggacaccaactttctgcccttgtcactcctgcctctgctgatgctccttgtccaaattggagcacagcctggggcccctgctcaaccacctgtgggctgggcatagccacccgagtgtccaaccagaaccgattctgccaactggagatccaacgccgcctgtgtctgcccagaccctgcctggcagccaggagccacagctcatggaacagtgctttctaa

SEQ ID NO: 4 - protein, 8 aa
  fragment type: internal
GCGCCXXC

SEQ ID NO: 5 - DNA (cDNA), 177 bp; single-stranded, linear
  CDS 1..177
tgtgaggtgaatggccgcaggtacctggatggagagacctttaaacccaattgcagggtcctgtgccgctgtgatgacggtggcttcacctgcctgccgctgtgcagtgaggatgtgcggctgcccagctgggactgcccacgccccaagagaatacaggtgccaggaaagtgctgc

SEQ ID NO: 6 - protein, 59 aa
CEVNGRRYLDGETFKPNCRVLCRCDDGGFTCLPLCSEDVRLPSWDCPRPKRIQVPGKCC

SEQ ID NO: 7 - protein, 15 aa
  fragment type: internal
WXXCSXXCGXGXXTR

SEQ ID NO: 8 - DNA (cDNA), 210 bp; single-stranded, linear
  CDS 1..210
cagctgtgccggacaccctgtacctgtccttggacaccaccccagtgcccacagggggtacccctggtgctggatggctgtggctgctgtaaagtgtgtgcacggaggctgggggagtcctgcgaccacctgcatgtctgcgaccccagccagggcctggtttgtcagcctggggcaggccctggcggccatggggctgtgtgtctcttg

SEQ ID NO: 9 - protein, 70 aa
QLCRTPCTCPWTPPQCPQGVPLVLDGCGCCKVCARRLGESCDHLHVCDPSQGLVCQPGAGPGGHGAVCLL

SEQ ID NO: 10 - DNA (cDNA), 174 bp; single-stranded, linear
  CDS 1..174
ccttgtccaaattggagcacagcctggggcccctgctcaaccacctgtgggctgggcatagccacccgagtgtccaaccagaaccgattctgccaactggagatccaacgccgcctgtgtctgcccagaccctgcctggcagccaggagccacagctcatggaacagtgctttc

SEQ ID NO: 11 - protein, 58 aa
PCPNWSTAWGPCSTTCGLGIATRVSNQNRFCQLEIQRRLCLPRPCLAARSHSSWNSAF

SEQ ID NO: 12 - DNA (cDNA), 681 bp; single-stranded, linear
  CDS 1..681
cagctgtgccggacaccctgtacctgtccttggacaccaccccagtgcccacagggggtacccctggtgctggatggctgtggctgctgtaaagtgtgtgcacggaggctgggggagtcctgcgaccacctgcatgtctgcgaccccagccagggcctggtttgtcagcctggggcaggccctggcggccatggggctgtgtgtctcttggatgaggatgacggtagctgtgaggtgaatggccgcaggtacctggatggagagacctttaaacccaattgcagggtcctgtgccgctgtgatgacggtggcttcacctgcctgccgctgtgcagtgaggatgtgcggctgcccagctgggactgcccacgccccaagagaatacaggtgccaggaaagtgctgccccgagtgggtatgtgaccagggagtgacaccggcgatccagcgctccacggcgcaaggacaccaactttctgcccttgtcactcctgcctctgctgatgctccttgtccaaattggagcacagcctggggcccctgctcaaccacctgtgggctgggcatagccacccgagtgtccaaccagaaccgattctgccaactggagatccaacgccgcctgtgtctgcccagaccctgcctggcagccaggagccacagctcatggaacagtgctttc

SEQ ID NO: 13 - protein, 227 aa
QLCRTPCTCPWTPPQCPQGVPLVLDGCGCCKVCARRLGESCDHLHVCDPSQGLVCQPGAGPGGHGAVCLLDEDDGSCEVNGRRYLDGETFKPNCRVLCRCDDGGFTCLPLCSEDVRLPSWDCPRPKRIQVPGKCCPEWVCDQGVTPAIQRSTAQGHQLSALVTPASADAPCPNWSTAWGPCSTTCGLGIATRVSNQNRFCQLEIQRRLCLPRPCLAARSHSSWNSAF

(19) United States
(12) Patent Application Publication
(10) Pub. No.: US 2002/0165185 A1
(43) Pub. Date: Nov. 7, 2002

(54) Novel Heparin-induced CCN-like molecules and uses therefor

(75) Inventor: John J. Castellot JR., Newton, MA (US)

Correspondence Address: LAHIVE & COCKFIELD, 28 STATE STREET, BOSTON, MA, 02109, US

(73) Assignee: Trustees of Tufts College

(21) Appl. No.: 10/010,408

(22) Filed: Dec. 7, 2001

Related U.S. Application Data
(63) Continuation of application No. 09/044,273, filed on Mar. 19, 1998, now abandoned.

(51) Int. Cl. A61K 48/00; C12Q 1/68; A01K 67/00; C12N 9/64; C07H 21/04

(52) U.S. Cl. 514/44; 800/8; 435/69.1; 435/226; 435/325; 435/320.1; 435/6; 536/23.2

(57) ABSTRACT

Novel HICP polypeptides, proteins, and nucleic acid molecules which play a role in cell proliferation and fibrosis are disclosed. In addition to isolated, full-length HICP proteins, the invention further provides isolated HICP fusion proteins, antigenic peptides and anti-HICP antibodies. The invention also provides HICP nucleic acid molecules, recombinant expression vectors containing a nucleic acid molecule of the invention, host cells into which the expression vectors have been introduced and non-human transgenic animals in which a HICP gene has been introduced or disrupted. Diagnostic, screening and therapeutic methods utilizing compositions of the invention are also provided.

### BACKGROUND OF THE INVENTION

In order to understand the mechanisms involved in cell proliferation and differentiation as well as to treat hyperproliferative disorders, efforts have focused on characterizing genes controlling cell growth and differentiation and genes encoding potential inhibitors of cell proliferation.

Several growth factors have been investigated to determine their role in stimulating cells to proliferate, differentiate and organize in developing tissues. Examples of some well-studied growth factors include platelet derived growth factor (PDGF), insulin-like growth factor (1GF-I), transforming growth factor beta (TGF-β) and connective tissue growth factor (CTGF). Studies of the roles of these growth factors have suggested that several of these factors may play a role, not only in the normal development, growth and repair of human tissue, but also may be involved pathologically in diseases or disorders characterized by uncontrolled tissue growth. For example, both PDGF and CTGF are known to be mitogens and chemotactic agents for connective tissue cells. In addition, both PDGF and CTGF have been implicated in disorders in which there is an overgrowth of connective tissue cells, such as cancer, fibrotic diseases and atheroclerosis.

Moreover, uncontrolled cell proliferation is involved in a number of other disease and disorders. For example, of the two million vascular procedures performed each year in the United States, approximately 500,000 of them fail within a few weeks due to smooth muscle cell hyperproliferation in the operated vessels.

Thus, there is a need to isolate genes encoding potential inhibitors of cell proliferation in order to develop diagnostics and therapeutics for various hyperproliferative disorders.

### SUMMARY OF THE INVENTION

The present invention provides a novel nucleic acid molecule which encodes a protein, referred to herein as Heparin-Induced, CCN-like protein (HICP), which is capable of modulating a variety of cellular processes including cell proliferation. Nucleic acid molecules encoding an HICP protein or polypeptide are referred to herein as HICP nucleic acid molecules. In a preferred embodiment, the HICP protein plays a role or functions in inhibiting or suppressing cell proliferation. In one aspect of the invention, a HICP protein is part of a signal transduction pathway in which HICP functions as part of an antiproliferation mechanism.

Heparin is known to inhibit or suppress proliferation in heparin-responsive cells, e.g., vascular smooth muscle cells (VSMC). The mechanism by which heparin inhibits proliferation in these cells is by binding to cell surface receptors on the cells. It is believed that heparin/receptor interaction results in selective modulation of the signal transduction pathway and altered transcription of a specific subset of growth regulatory genes. HICP expression is upregulated in heparin-treated cells and plays a role in the antiproliferative mechanism of action of heparin. Thus, HICP molecules of the present invention can be used to modulate proliferation of heparin-responsive cells (e.g., VSMC) and thus to treat proliferative disorders such as cardiovascular disorders.

In addition, the HICP molecules of the present invention to play a role in growth factor signaling pathways, e.g., CTGF signal pathway. The HICP molecules of the present invention are capable of interfering with growth factor signaling to thereby inhibit or suppress growth factor induced proliferation, cell motility and extracellular matrix production. Thus, HICP molecules can also be used to modulate cell proliferation, cell motility and extracellular matrix production in various cell types and thus can be used to treat other disorders characterized by aberrant or abnormal cell proliferation and fibroproliferative disorders, e.g., fibrotic disorders.

Accordingly, in one aspect, this invention provides isolated nucleic acid molecules encoding HICP proteins or biologically active portions thereof, as well as nucleic acid fragments suitable as primers or hybridization probes for the detection of HICP-encoding nucleic acids.

In one embodiment, a HICP nucleic acid molecule is 60% homologous to the nucleotide sequence shown in SEQ ID NO:1, SEQ ID NO:3 or SEQ ID NO:12. In a preferred embodiment, an isolated HICP nucleic acid molecule encodes the amino acid sequence of rat HICP. In another embodiment, a HICP nucleic acid molecule is at least 60-70% or more homologous to the nucleotide sequence shown in SEQ ID NO: 1, SEQ ID NO:3 or SEQ ID NO: 12 and encodes a protein with one or more of the following activities: 1) it can modulate cell proliferation; 2) it can modulate a growth factor signaling pathway; 3) it can modulate the activity of CTGF or PDGF; 4) it can modulate a heparin-induced response in a heparin-responsive cell (e.g., a VSMC); 5) it can modulate cell motility; 6) it can modulate extracellular matrix production.

In a preferred embodiment, an isolated HICP nucleic acid molecule has the nucleotide sequence of SEQ ID NO:1 or a complement thereof. In another embodiment, a HICP nucleic acid molecule further comprises nucleotides 1-883 of SEQ ID NO:1.

In yet another preferred embodiment of the invention, an isolated HICP nucleic acid molecule has the nucleotide sequence of SEQ ID NO:3 or a complement thereof. In another embodiment, a HICP nucleic acid molecule further comprises nucleotides 1-635 of SEQ ID NO:3. In a further embodiment of the invention, an isolated HICP nucleic acid molecule has the nucleotide sequence of SEQ ID NO: 10 or a complement thereof. In another embodiment, a HICP nucleic acid molecule further comprises nucleotides 1-612 of SEQ ID NO:12.

In another embodiment, a HICP nucleic acid molecule includes a nucleotide sequence encoding a protein having an amino acid sequence sufficiently homologous to the amino acid sequence of SEQ ID NO:2 or SEQ ID NO:13. In yet another embodiment, a HICP nucleic acid molecule includes a nucleotide sequence encoding a protein having an amino acid sequence at least 60% homologous to the amino acid sequence of SEQ ID NO:2 or SEQ ID NO:13. In a preferred embodiment, a HICP nucleic acid molecule includes a nucleotide sequence encoding a protein having the amino acid sequence of SEQ ID NO: 2. In another preferred embodiment, a HICP nucleic acid molecule includes a nucleotide sequence encoding a protein having the amino acid sequence of SEQ ID NO: 13.

In another embodiment, an isolated nucleic acid molecule of the present invention encodes a HICP protein which includes at least one, preferably two or three modular domains selected from the group consisting of an insulin-like growth factor binding protein (IGFBP) domain, a Von Willebrand C (VWC) domain and a thrombospondin 1 (TSP 1) domain. In another embodiment, the HICP nucleic acid molecule encodes a protein which includes at least one modular domain, a signal sequence and is cysteine-rich. In another embodiment, the HICP nucleic acid molecule encodes a HICP protein and is a naturally occurring nucleotide sequence. In yet another embodiment, an isolated nucleic acid molecule of the present invention encodes a HICP protein and comprises a nucleotide sequence which hybridizes under stringent hybridization conditions to a nucleic acid molecule having the nucleotide sequence of SEQ ID NO:1, SEQ ID NO:3 or SEQ ID NO:12.

Another embodiment of the invention features HICP nucleic acid molecules which specifically detect HICP nucleic acid molecules relative to nucleic acid molecules encoding non-HICP proteins. For example, in one embodiment, a HICP nucleic acid molecule hybridizes under stringent conditions to a nucleic acid molecule comprising nucleotides 1-883 of SEQ ID NO: 1. In another exemplary embodiment, a HICP nucleic acid molecule hybridizes under stringent conditions to a nucleic acid molecule comprising nucleotides 1-635 of SEQ ID NO:3. In yet another exemplary embodiment, a HICP nucleic acid molecule hybridizes under stringent conditions to a nucleic acid molecule comprising nucleotides 1-612 of SEQ ID NO:12. In another embodiment, the HICP nucleic acid molecule is at least 500 nucleotides in length and hybridizes under stringent conditions to a nucleic acid molecule comprising the nucleotide sequence shown in SEQ ID NO:1, SEQ ID NO:3, SEQ ID NO:12 or a complement thereof Another embodiment the invention provides an isolated nucleic acid molecule which is antisense to the coding strand of a HICP nucleic acid.

Another aspect of the invention provides a vector comprising a HICP nucleic acid molecule. In certain embodiments, the vector is a recombinant expression vector. In another embodiment the invention provides a host cell containing a vector of the invention. The invention also provides a method for producing HICP protein by culturing in a suitable medium, a host cell of the invention containing a recombinant expression vector such that HICP protein is produced.

Another aspect of this invention features isolated or recombinant HICP proteins and polypeptides. In a preferred embodiment, the HICP protein or polypeptide can modulate cell proliferation. In another embodiment, an isolated HICP protein has an amino acid sequence sufficiently homologous to the amino acid sequence of SEQ ID NO:2. or SEQ ID NO:13, e.g., sufficiently homologous to an amino acid sequence of SEQ ID NO:2 or SEQ ID NO: 13 such that the protein or polypeptide maintains an HICP activity. In a preferred embodiment, a HICP protein has an amino acid sequence at least about 60% homologous to the amino acid sequence of SEQ ID NO:2 or SEQ ID NO: 13. In another embodiment, a HICP protein has the amino acid sequence of SEQ ID NO:2 or SEQ ID NO: 13. In yet another embodiment, the isolated HICP protein comprises an amino acid sequence which is at least about 60-70% or more homologous to the amino acid sequence of SEQ ID NO:2 or SEQ ID NO:13 and has one or more of the following activities: 1) it can modulate cell proliferation; 2) it can modulate a growth factor signaling pathway; 3) it can modulate the activity of CTGF or PDGF; 4) it can modulate a heparin-induced response in a heparin-responsive cell (e.g., a VSMC); 5) it can modulate cell motility; 6) it can modulate extracellular matrix production.

In another embodiment, an isolated HICP protein has at least one, preferably two or three, modular domains selected from an IGFBP domain, a VWC domain and a TSP 1 domain. In yet another embodiment, an isolated HICP protein has at least one modular domain, a signal sequence and is cysteine-rich.

Another embodiment of the invention features an isolated HICP protein which is encoded by a nucleic acid molecule having a nucleotide sequence at least about 60% homologous to a nucleotide sequence of SEQ ID NO:1, SEQ ID NO:3, SEQ ID NO:12 or a complement thereof. This invention also features an isolated HICP protein which is encoded by a nucleic acid molecule having a nucleotide sequence which hybridizes under stringent hybridization conditions to a nucleic acid molecule comprising the nucleotide sequence of SEQ ID NO:1, SEQ ID NO:3, SEQ ID NO:12, or a complement thereof.

The HICP proteins of the present invention, or biologically active portions thereof, can be operatively linked to a non-HICP polypeptide to form HICP fusion proteins. The invention further features antibodies that specifically bind HICP proteins, such as monoclonal or polyclonal antibodies. In addition, the HICP proteins or biologically active portions thereof can be incorporated into pharmaceutical compositions, which optionally include pharmaceutically acceptable carriers.

In another aspect, the present invention provides a method for detecting HICP expression in a biological sample by contacting the biological sample with an agent capable of detecting a HICP nucleic acid molecule, protein or polypeptide such that the presence of HICP nucleic acid molecule, protein or polypeptide is detected in the biological sample.

In another aspect, the present invention provides a method for detecting the presence of HICP activity in a biological sample by contacting the biological sample with an agent capable of detecting an indicator of HICP activity such that the presence of HICP activity is detected in the biological sample.

In another aspect, the invention provides a method for modulating HICP activity comprising contacting the cell with an agent that modulates HICP activity such that HICP activity in the cell is modulated. In one embodiment, the agent inhibits HICP activity. In another embodiment, the agent stimulates HICP activity. In one embodiment, the agent is an antibody that specifically binds to HICP protein. In another embodiment, the agent modulates expression of HICP by modulating transcription of a HICP gene or translation of a HICP mRNA. In yet another embodiment, the agent is a nucleic acid molecule having a nucleotide sequence that is antisense to the coding strand of the HICP mRNA or the HICP gene.

In one embodiment, the methods of the present invention are used to treat a subject having a disorder characterized by abnormal or aberrant cell proliferation or fibroproliferation by administering a HICP agent to the subject. In one embodiment, the HICP agent is a HICP protein or polypeptide. In another embodiment, the HICP agent is a nucleic acid molecule encoding a HICP protein. In a preferred embodiment, the disorder characterized by abnormal cell proliferation is a fibrotic disorder or a cardiovascular disorder. In another preferred embodiment, the disorder characterized by abnormal fibroproliferation is a fibrotic disorder such as abnormal scarring, fibrosis or keloidosis.

The present invention also provides a diagnostic assay for identifying the presence or absence of a genetic alteration characterized by at least one of (i) aberrant modification or mutation of a gene encoding a HICP protein; (ii) mis-regulation of said gene; and (iii) aberrant post-translational modification of a HICP protein, wherein a wild-type form of said gene encodes an protein with a HICP activity.

Another aspect the invention provides a method for identifying a compound that binds to or modulates the activity of a HICP protein, by providing a indicator composition comprising a HICP protein having HICP activity, contacting the indicator composition with a test compound, and determining the effect of the test compound on HICP activity in the indicator composition to identify a compound that modulates the activity of a HICP protein.

In yet another aspect, the invention provides a method for identifying and isolating heparin species which are involved in antiproliferation activity of heparin-responsive cells, by providing a HICP protein or polypeptide which acts as an affinity reagent or screening reagent for isolating specific heparin species involved in anti-proliferation activity.

Other features and advantages of the invention will be apparent from the following detailed description and claims.

### BRIEF DESCRIPTION OF THE DRAWINGS

FIG. 1 depicts the cDNA sequence and predicted amino acid sequences of rat HICP. The nucleotide sequence corresponds to nucleic acids 1 to 1708 of SEQ ID NO:1. The amino acid sequence corresponds to amino acids 1 to 250 of SEQ ID NO:2.

FIG. 2 depicts the cDNA sequence and predicted amino acid sequences of mature rat HICP. The nucleotide sequence corresponds to nucleic acids 1 to 681 of SEQ ID NO:12. The amino acid sequence corresponds to amino acids 1 to 227 of SEQ ID NO:13.

FIG. 3 depicts an alignment of the amino acid sequences of the modular domains of rat HICP and with the insulin-like growth factor binding protein (IGFBP) domain, von Willibrand Factor C (VWC) domain and the thrombospondin 1 (TSP1) domain of CTGF, CEF-10 and NOV.

FIG. 4 depicts the kinetics of HICP mRNA expression in quiescent rat aorta smooth muscle cells (RASMC) treated with fetal calf serum (F) in the absence and presence of heparin (H). H* represents a delayed addition experiment, in which heparin was added after four hours of serum stimulation.

FIG. 5 depicts DNA synthesis in rat aorta smooth muscle cells after exposure to conditioned medium from HICP transfected COS cells (HICP-CM), conditioned medium from control COS cells transfected with vector alone (Vector-CM), or fetal calf serum (FCS). DNA synthesis is measured in counts per minute (CPM) using scintillation counting.

### DETAILED DESCRIPTION OF THE INVENTION

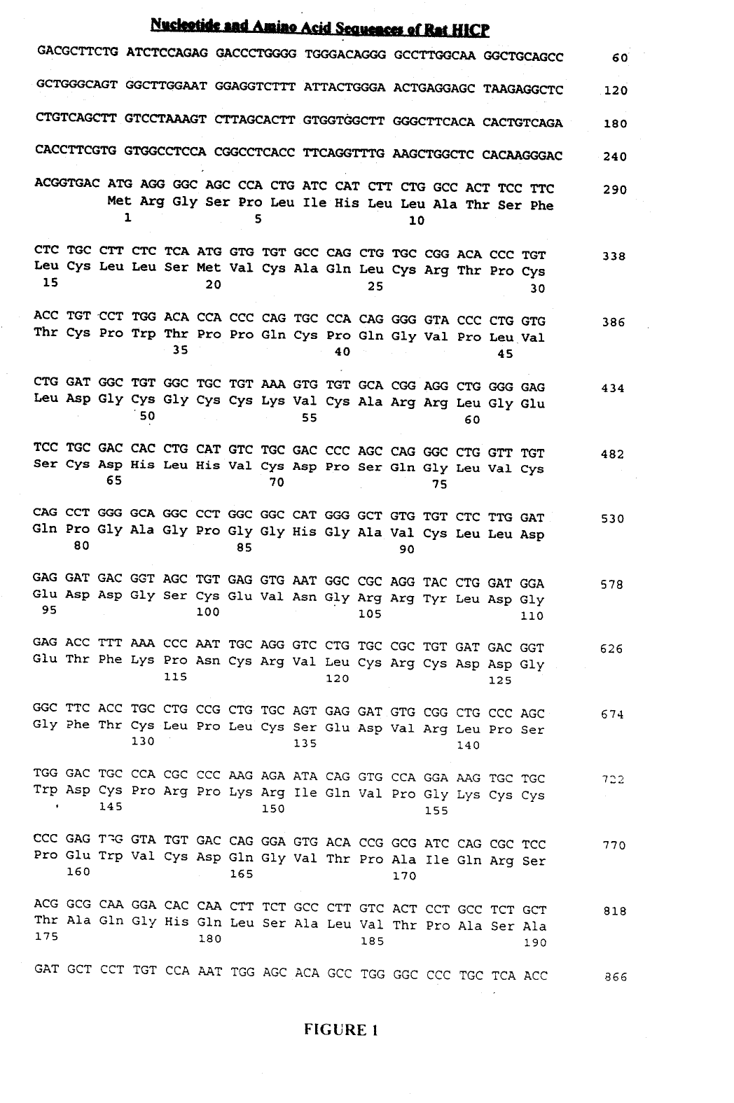

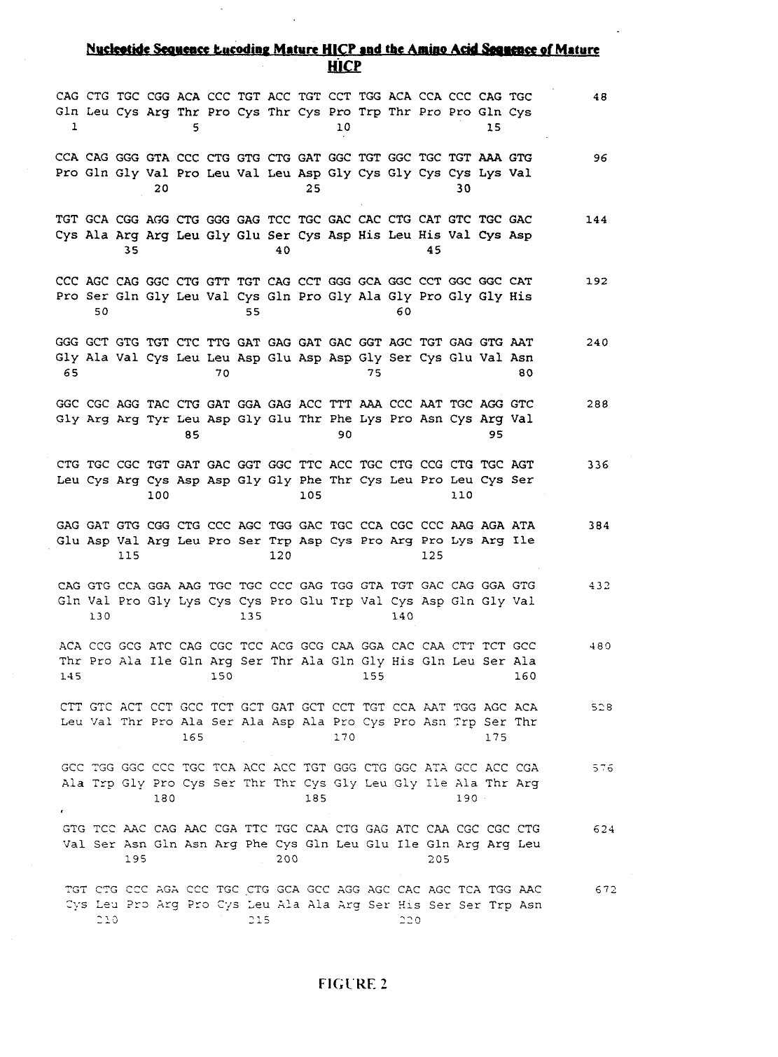

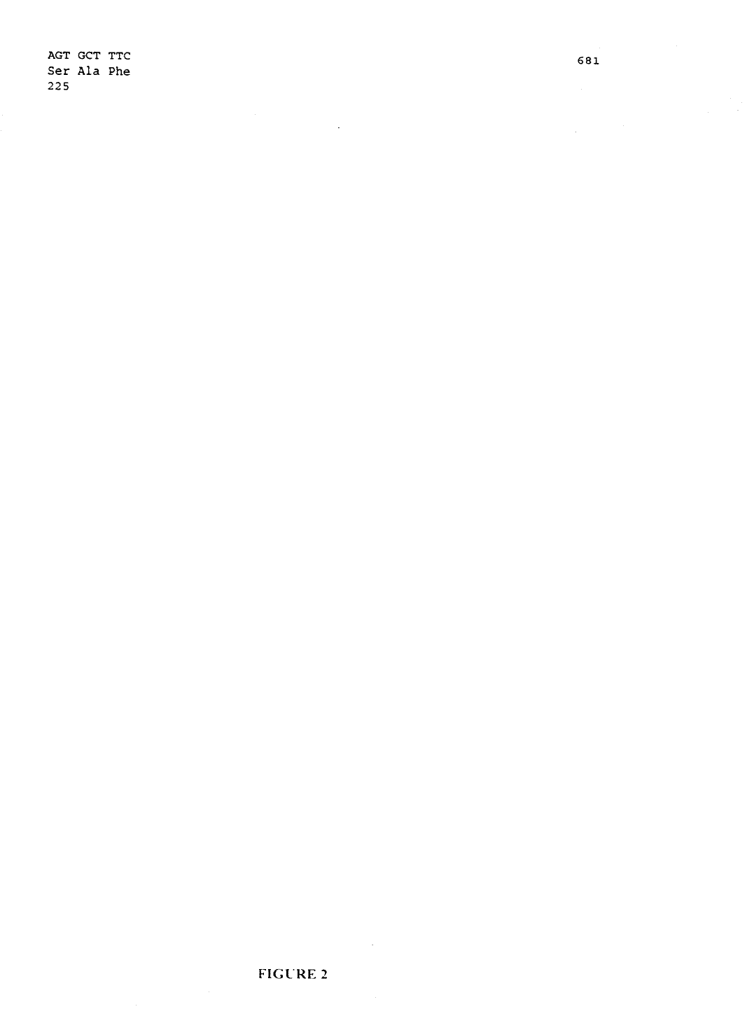

The present invention is based on the discovery of novel molecules, referred to herein as HICP protein and nucleic acid molecules, which play a role in or function in cell proliferation, cell motility and extracellular matrix production and have certain conserved structural features. The nucleotide sequences of rat HICP nucleic acid molecule and the amino acid sequence of the rat HICP protein molecule are depicted in FIG. 1.

In one embodiment, the HICP molecules modulate cell proliferation by playing a role in growth inhibition in heparin responsive cells. In a preferred embodiment, HICP modulates (e.g., mediates) the antiproliferative effect of heparin. Heparin has been shown to suppress proliferation of various cell types, e.g., vascular smooth muscle cells (VSMC). It is believed that the mechanism by which heparin inhibits proliferation of cells is by binding to receptors on the cell surface which leads to selective modulation of signal transduction pathways and altered transcription of specific growth regulatory genes. HICP proteins are expressed at increased levels in heparin-treated cells, and play a role in the antiproliferative effect of heparin on heparin-responsive cells. Thus, the HICP molecules are capable of modulating proliferation of heparin-responsive cells, e.g., VSMC. Thus, the HICP molecules of the present invention can be used to treat various cardiovascular diseases or disorders such as restenosis, ischemia and atherosclerosis.

In another embodiment, the HICP molecules are capable of modulating the activity of one or more proteins involved in a growth factor signaling pathway, e.g., a CTGF signaling pathway. In a preferred embodiment, the HICP molecules modulate the activity of one or more proteins by interfering with or preventing signal transduction. For example, a HICP molecule can interfere with the activity or one or more proteins involved in a growth factor signaling pathway by acting as a growth factor antagonist, thereby inhibiting or suppressing a growth factor induced activity, e.g., proliferation, cell motility or extracellular matrix production. CTGF is known to be a mitogen, a chemotactic agent for connective tissue and a stimulator of extracellular matrix production. In addition, this growth factor has been implicated as a major factor involved diseases and disorders characterized by hyperproliferation of connective tissue cells. Such diseases include, for example, atherosclerosis and fibrotic diseases. See Grotendorst et al., U.S. Pat. No. 5,408,040 and Grotendorst et al., U.S. Pat. No. 5,585,270. Thus, the HICP protein, by acting as a growth factor antagonist, e.g., a CTGF antagonist, can modulate (e.g., inhibit) aberrant or abnormal cell proliferation, cell motility and/or extracellular matrix production. Thus, HICP molecules (or modulators thereof) of the present invention can be used to treat various fibroproliferative disorders in which fibrosis is an important feature of the pathology, e.g., abnormal scarring, keloidosis and kidney and lung fibrosis.

As used interchangeably herein an “HICP activity”, “biological activity of HICP” or “functional activity of HICP”, refers to an activity exerted by a HICP protein, polypeptide or nucleic acid molecule on a HICP responsive cell as determined in vivo, or in vitro, according to standard techniques. In one embodiment, a HICP activity is a direct activity, such as direct mediation of cellular proliferation. In another embodiment, a HICP activity is an indirect activity, such as the interference by a HICP protein with a signaling pathway. In a preferred embodiment, a HICP activity is at least one or more of the following activities: (i) interaction of a HICP protein in the extracellular milieu with a non-HICP protein molecule on the surface of the same cell which secreted the HICP protein molecule; (ii) interaction of a HICP protein in the extracellular milieu with a non-HICP protein molecule on the surface of a different cell from that which secreted the HICP protein molecule; (iii) complex formation between a HICP protein and a HICP receptor; (iv) complex formation between a HICP protein and non-HICP receptor; and (v) interaction of a HICP protein with a second protein in the extracellular milieu. In yet another preferred embodiment, a HICP activity is at least one or more of the following activities: (i) modulation of cell proliferation; (ii) modulation of a growth factor signaling pathway; (iii) modulation of the activity of CTGF; (iv) modulation of a heparin-induced response in a heparin-responsive cell (e.g., a VSMC); (v) modulation of cell motility; (vi) modulation of extracellular matrix production.

In another aspect of the invention, the HICP nucleic acid and protein molecules have homology to members of the CCN protein family, which comprise a family of molecules having certain conserved structural and functional features. The term “family” when referring to the protein and nucleic acid molecules of the invention is intended to mean two or more protein or nucleic acid molecules having a common structural domain and having sufficient amino acid or nucleotide sequence homology as defined herein. Such family members can be naturally occurring and can be from either the same or different species. For example, a family can contain a first protein of human origin and a homologue of that protein of rat origin, as well as a second, distinct protein of human origin and a rat homologue of that protein. Members of a family may also have common functional characteristics. An exemplary family of the present invention is the CCN family whose members include, at least, CTGF, Cyr61, CEF-10 and Nov.

In one embodiment, an HICP family member is identified based on the presence of at least one, preferably two or three, modular domains. The term “modular domains”, as used herein, refers to a group of three domains which are conserved among HICP family members and also CCN family members. These three domains are an insulin-like growth factor binding protein domain, a Von Willebrand C domain and a thrombospondin 1 domain.

In one embodiment, a HICP family member is identified based on the presence of at least one “insulin-like growth factor binding protein domain” (also referred to herein as an “IGFBP domain”) in the protein or corresponding nucleic acid molecule. As used herein, the term “IGFBP domain” refers to a protein domain which is at least about 50-90 amino acid residues in length, preferably at least about 60-80 amino acid residues in length, and more preferably at least about 70-75 amino acid residues in length, and has at least about 30-50%, preferably as least about 35-55% homology with the amino acid sequence of rat HICP, as set forth in SEQ ID NO:9. In a preferred embodiment, the IGFBP domain is a cysteine-rich domain which includes preferably 9, more preferably 10-11, and even more preferably 12 cysteine residues.

In addition, an IGFBP domain has an “insulin-like growth factor binding protein motif” (also referred to herein as an “IGFBP motif”). The term “IGFBP motif”, as used herein, refers to a conserved motif of a HICP family member (or a CCN family member) which includes about eight amino acid residues. An IGFBP motif comprises the following amino acid sequence: G-C-G-C-C-X-X-C (SEQ ID NO:4) wherein X is any amino acid residue. In one embodiment, a HICP protein includes an IGFBP motif having at least about 60%, preferably at least 65% to about 70%, and more preferably about 75% amino acid sequence homology to an IGFBP motif having amino acid residues 49-56 of SEQ ID NO:2. In a preferred embodiment, a HICP protein has the IGFBP motif of amino acid residues 49-56 of SEQ ID NO:2.

In another embodiment, a HICP family member is identified based on the presence of at least one “Von Willebrand C domain” (also referred to herein as a “VWC domain”) in the protein or corresponding nucleic acid molecule. As used herein, the term “VWC domain” refers to a protein domain which is at least about 40-90 amino acid residues in length, preferably at least about 55-85 amino acid residues in length, and more preferably at least about 70-80 amino acid residues in length, and has at least about 30-60%, preferably at least about 35-55%, more preferably at least about 30-45% homology with the amino acid sequence of rat HICP, as set forth in SEQ ID NO:6. In a preferred embodiment, the VWC domain is a cysteine-rich domain which includes preferably 6-7, more preferably 8-9, and even more preferably, 10 cysteine residues.

In yet another embodiment, a HICP family member is identified based on the presence of at least one “thrombospondin 1 domain” (also referred to herein as a “TSP1 domain”) in the protein or corresponding nucleic acid molecule. As used herein, the term “TSP1 domain” refers to a protein domain which is at least about 40-80 amino acid residues in length, preferably at least about 50-70 amino acid residues in length, and more preferably at least about 60 amino acid residues in length, and has at least about 30-60%, preferably as least about 40-55% homology with the amino acid sequence of rat HICP, as set forth in SEQ ID NO:1 1. In a preferred embodiment, the TSP1 domain is a cysteine-rich domain which includes preferably 4, more preferably 5, and even more preferably 6 cysteine residues.

In addition, a TSP1 domain has a “thrombospondin 1 motif” (also referred to herein as a “TSP1 motif”). The term “TSP1 motif”, as used herein, refers to a conserved motif of a HICP family member (or a CCN family member) which includes a heparin binding motif and is at least about 15-20 amino acid residues, preferably about 17-19 amino acid residues, and more preferably about 18 amino acid residues in length. A TSP 1 domain preferably includes the following amino acid sequence motif: W-X-X-C-S-X-X-C-G-X-G-X-X-T-R (SEQ ID NO:7) wherein X is any amino acid residue. In one embodiment, a HICP protein includes a TSP 1 motif having at least about 60%, preferably at least 65% to about 70%, and more preferably about 75% amino acid sequence homology to a TSP 1 motif having amino acid residues 201-215 of SEQ ID NO:2. In a preferred embodiment, a HICP protein has the TSP1 motif of amino acid residues 201-215 of SEQ ID NO:2.

Another embodiment of the invention features HICP molecules which contain a signal sequence. As used herein, a “signal sequence” refers to a peptide containing about 23 amino acids which occurs at the extreme N-terminal end of secretory proteins and which contains large numbers of hydrophobic amino acid residues. For example, a signal sequence contains at least about 15-30 amino acid residues, preferably about 17-28 amino acid residues, more preferably about 19-26 amino acid residues, and more preferably about 20-25 amino acid residues, and has at least about 40-70%, preferably about 45-60%, and more preferably about 50-55% hydrophobic amino acid residues (e.g., Alanine, Valine, Leucine, Isoleucine, Phenylalanine, Tyrosine, Tryptophan, or Proline). Such a “signal sequence”, also referred to in the art as a “signal peptide”, serves to direct a protein containing such a sequence to a lipid bilayer and usually means that the protein will be secreted.

In yet another embodiment, a HICP protein is cysteine-rich. As used herein the term “cysteine-rich” refers to an HICP protein which contains at least about 20-40, more preferably at least 25-35, and even more preferably at least 28 cysteine residues. For example, in one embodiment, a HICP protein contains a cysteine residues at amino acid residue 16, 22, 26, 30, 32, 39, 50, 56, 64, 70, 78, 91, 100, 117, 121, 123, 130, 134, 145, 157, 158, 163, 194, 204, 208, 223, 232, and/or amino acid residue 237 of SEQ ID NO: 2.

In a preferred embodiment, a HICP protein contains at least one, preferably two or three, modular domains and is cysteine rich. In a preferred embodiment, a HICP protein further contains a signal sequence. In one exemplary embodiment, a HICP protein contains an IGFBP domain including an IGFBP motif comprising amino acids 49-56 of SEQ ID NO:2, a VWC domain comprising amino acid residues 100-158 of SEQ ID NO:2 and/or a TSP1 domain including a TSP1 motif comprising amino acid residues 201-215 of SEQ ID NO:2 and further contains about 28 cysteine residues. In another exemplary embodiment, a HICP protein contains further contains a signal sequence at about amino acids 1-23 of SEQ ID NO:2.

In yet another embodiment, a HICP protein encodes a mature HICP protein. As used herein, the term “mature HICP protein” refers to a HICP protein from which the signal peptide has been cleaved. In an exemplary embodiment, a mature HICP protein contains amino acid residues 1 to 227 of SEQ ID NO: 13. In a preferred embodiment, a HICP protein is a mature HICP protein which contains at least one, preferably two or three, modular domain and is cysteine rich.

Preferred HICP molecules of the present invention have an amino acid sequence sufficiently homologous to the amino acid sequence of SEQ ID NO:2 or SEQ ID NO:13. As used herein, the term “sufficiently homologous” refers to a first amino acid or nucleotide sequence which contains a sufficient or minimum number of identical or equivalent (e.g., an amino acid residue which has a similar side chain) amino acid residues or nucleotides to a second amino acid or nucleotide sequence such that the first and second amino acid or nucleotide sequences share common structural domains and/or a common functional activity. For example, amino acid or nucleotide sequences which share common structural domains have at least about 40% homology, preferably 50% homology, more preferably 60%-70% homology across the amino acid sequences of the domains and contain at least one, preferably two, and more preferably three or four structural domains, are defined herein as sufficiently homologous. Furthermore, amino acid or nucleotide sequences that share at least 40%, preferably 50%, more preferably 60, 70, or 80% homology and share a common functional activity are defined herein as sufficiently homologous.

In a particularly preferred embodiment, the HICP protein and nucleic acid molecules of the present invention are rat HICP molecules. A rat HICP cDNA molecule was obtained from a rat subtraction cDNA library which was enriched for sequences in fetal calf serum (FCS) and heparin treated vascular smooth muscle cells (VSMC) versus VSMC treated only with FCS as described in Example 1. The nucleotide sequence of the isolated rat HICP cDNA and the predicted amino acid sequence of the rat HICP protein are shown in FIG. 1 and in SEQ ID NOs: 1 and 2, respectively. In addition, the nucleotide sequences corresponding to the coding region of the rat HICP cDNA and the HICP cDNA encoding the mature HICP protein are represented as SEQ ID NO:3 and SEQ ID NO:13, respectively.

Because of the ability of heparin, which suppresses cell proliferation in heparin responsive cells, to specifically induce HICP cDNA expression, the HICP molecules of the present invention also play a role in heparin-induced antiproliferation mechanisms.

HICP mRNA is also expressed in other tissues besides vascular smooth muscle cells. A HICP mRNA transcript of approximately 1.8 to 1.9 kb was expressed in normal uninjured adult rat aorta (See Example 2). HICP was also expressed in high levels in lung, heart brain and skeletal muscles. Little or no expression of HICP is found in the spleen, liver, kidney or testes.

The rat HICP cDNA set forth in SEQ ID NO: 1, is approximately 1708 nucleotides in length and encodes a protein which is approximately 250 amino acid residues in length (SEQ ID NO:2). The rat HICP protein contains a signal sequence, an IGFBP domain with an IGFBP motif, a VWC motif and a TSP1 domain with a TSP1 motif, as defined herein. A HICP-IGFBP motif can be found at least, for example, from about amino acids 49-56 of SEQ ID NO:2. A VWC motif can be found at least, for example, from about amino acids 101-165 of SEQ ID NO:2 and a TSP1 motif can be found at least, for example, from about amino acids 201-215 of SEQ ID NO:2. A signal sequence can be found at least, for example, from about amino acids 1-23 of SEQ ID NO:2.

A GeneBank™ search using rat HICP nucleotide and amino acid sequences revealed nucleotide sequence similarity with nucleotide sequences of several members of the CCN family including Cyr61, Nov and CTGF and amino acid sequence similarities with CTGF from several different species. For example, a BLASTPTM search using the rat HICP amino acid sequence of SEQ ID NO:2 identified a 53% sequence identity between amino acid residues 95 to 165 of SEQ ID NO:2 and both murine CTGF (Accession Number P29268) and human CTGF (Accession Number P29279). (The alignment was generated using MegAlign™ sequence alignment software. The initial pairwise alignment step was performed using a Wilbur-Lipmann algorithm with a K-tuple of 2, a GAP penalty of 5, a window of 4, and diagonals saved set to =4. The multiple alignment step was performed using the Clustal algorithm with a PAM 250 residue weight Table, a GAP penalty of 10, and a GAP length penalty of 10.) Various aspects of the invention are described in further detail in the following subsections:

I. Isolated Nucleic Acid Molecules

One aspect of the invention pertains to isolated nucleic acid molecules that encode HICP proteins or biologically active portions thereof, as well as nucleic acid fragments sufficient for use as hybridization probes to identify HICP-encoding nucleic acids (e.g., HICP mRNA) and fragments for use as PCR primers for the amplification or mutation of HICP nucleic acid molecules. As used herein, the term “nucleic acid molecule” is intended to include DNA molecules (e.g., cDNA or genomic DNA) and RNA molecules (e.g., mRNA) and analogs of the DNA or RNA generated using nucleotide analogs. The nucleic acid molecule can be single-stranded or double-stranded, but preferably is double-stranded DNA.

An “isolated” nucleic acid molecule is one which is separated from other nucleic acid molecules which are present in the natural source of the nucleic acid. Preferably, an “isolated” nucleic acid is free of sequences which naturally flank the nucleic acid (i.e., sequences located at the 5′ and 3′ ends of the nucleic acid) in the genomic DNA of the organism from which the nucleic acid is derived. For example, in various embodiments, the isolated HICP nucleic acid molecule can contain less than about 5 kb, 4 kb, 3 kb, 2 kb, 1 kb, 0.5 kb or 0.1 kb of nucleotide sequences which naturally flank the nucleic acid molecule in genomic DNA of the cell from which the nucleic acid is derived. Moreover, an “isolated” nucleic acid molecule, such as a cDNA molecule, can be substantially free of other cellular material, or culture medium when produced by recombinant techniques, or substantially free of chemical precursors or other chemicals when chemically synthesized.

A nucleic acid molecule of the present invention, e.g., a nucleic acid molecule having the nucleotide sequence of SEQ ID NO:1, SEQ ID NO:3, SEQ ID NO:12, or a portion thereof, can be isolated using standard molecular biology techniques and the sequence information provided herein. Using all or portion of the nucleic acid sequence of SEQ ID NO:1, SEQ ID NO:3 or SEQ ID NO:12 as a hybridization probe, HICP nucleic acid molecules can be isolated using standard hybridization and cloning techniques (e.g., as described in Sambrook, J., Fritsh, E. F., and Maniatis, T. Molecular Cloning: A Laboratory Manual. 2^{nd}, ed, Cold Spring Harbor Laboratory, Cold Spring Harbor Laboratory Press, Cold Spring Harbor, N.Y., 1989). In another embodiment, a portion of the nucleic acid sequence of SEQ ID NO:1, from nucleotide 1 to 883 or nucleotide 1533 to 1708, can used as a hybridization probe.

Moreover, a nucleic acid molecule encompassing all or a portion of SEQ ID NO:1, SEQ ID NO:3 or SEQ ID NO:12 can be isolated by the polymerase chain reaction (PCR) using synthetic oligonucleotide primers designed based upon the sequence of SEQ ID NO:1, SEQ ID NO:3, or SEQ ID NO:12.

A nucleic acid of the invention can be amplified using cDNA, mRNA or alternatively, genomic DNA, as a template and appropriate oligonucleotide primers according to standard PCR amplification techniques. The nucleic acid so amplified can be cloned into an appropriate vector and characterized by DNA sequence analysis. Furthermore, oligonucleotides corresponding to HICP nucleotide sequences can be prepared by standard synthetic techniques, e.g., using an automated DNA synthesizer.

In a preferred embodiment, an isolated nucleic acid molecule of the invention comprises the nucleotide sequence shown in SEQ ID NO: 1. The sequence of SEQ ID NO:1 corresponds coding and noncoding regions of rat HICP cDNA. This cDNA comprises sequences encoding the rat HICP protein (i.e., “the coding region”, from nucleotides 249-998) and noncoding regions (i.e., from nucleotides 1-248 and from nucleotides 999-1708).

In yet another embodiment, an isolated nucleic acid molecule of the invention comprises the nucleotide sequence shown in SEQ ID NO:3. The sequence of SEQ ID NO:3 corresponds to rat HICP cDNA. This cDNA comprises sequences encoding the rat HICP protein (i.e., “the coding region”, from nucleotides 249-998 of SEQ ID NO:1).

In further embodiment, an isolated nucleic acid molecule of the invention comprises the nucleotide sequence shown in SEQ ID NO:12. The sequence of SEQ ID NO:12 corresponds to rat HICP cDNA. This CDNA comprises sequences encoding the mature rat HICP protein (i.e., from nucleotides 318-998 of SEQ ID NO:1).

In another preferred embodiment, an isolated nucleic acid molecule of the invention comprises a nucleic acid molecule which is a complement of the nucleotide sequence shown in SEQ ID NO:1, SEQ ID NO:3, SEQ ID NO:12 or a portion of either of these nucleotide sequences. A nucleic acid molecule which is complementary to the nucleotide sequence shown in SEQ ID NO:1, SEQ ID NO:3 or SEQ ID NO:12 is one which is sufficiently complementary to the nucleotide sequence shown in SEQ ID NO:1, SEQ ID NO:3 or SEQ ID NO:12 such that it can hybridize to the nucleotide sequence shown in SEQ ID NO:1, SEQ ID NO:3 or SEQ ID NO:12, thereby forming a stable duplex.

In still another preferred embodiment, an isolated nucleic acid molecule of the present invention comprises a nucleotide sequence which is at least about 60-65%, preferably at least about 70-75%, more preferable at least about 80-85%, and even more preferably at least about 90-95% or more homologous to the nucleotide sequences shown in SEQ ID NO:1, SEQ ID NO:3, or SEQ ID NO: 12, or a portion of any of these nucleotide sequences.

Moreover, the nucleic acid molecule of the invention can comprise only a portion of the nucleic acid sequence of SEQ ID NO:1, SEQ ID NO:3 or SEQ ID NO:12, for example a fragment which can be used as a probe or primer or a fragment encoding a biologically active portion of a HICP protein. The nucleotide sequence determined from the cloning of the murine HICP genes allows for the generation of probes and primers designed for use in identifying and/or cloning HICP homologues in other cell types, e.g., from other tissues, as well as HICP homologues from other mammals. The probe/primer typically comprises substantially purified oligonucleotide. The oligonucleotide typically comprises a region of nucleotide sequence that hybridizes under stringent conditions to at least about 12, preferably about 25, more preferably about 40, 50 or 75 consecutive nucleotides of a sense sequence of SEQ ID NO: 1, SEQ ID NO:3 or SEQ ID NO: 12 of an anti-sense sequence of SEQ ID NO:1, SEQ ID NO:3, SEQ ID NO:12 or of a naturally occurring mutant of SEQ ID NO:1, SEQ ID NO:3 or SEQ ID NO:12. In an exemplary embodiment, a nucleic acid molecule of the present invention comprises a nucleotide sequence which hybridizes under stringent hybridization conditions to a nucleic acid molecule comprising the nucleotide sequence of SEQ ID NO: 1, SEQ ID NO:3 or SEQ ID NO:12.

Probes based on the rat HICP nucleotide sequence can be used to detect transcripts or genomic sequences encoding the same or homologous proteins. In preferred embodiments, the probe further comprises a label group attached thereto, e.g., the label group can be a radioisotope, a fluorescent compound, an enzyme, or an enzyme co-factor. Such probes can be used as a part of a diagnostic test kit for identifying cells or tissue which misexpress a HICP protein, such as by measuring a level of a HICP-encoding nucleic acid in a sample of cells from a subject e.g., detecting HICP mRNA levels or determining whether a genomic HICP gene has been mutated or deleted.

A nucleic acid fragment encoding a “biologically active portion of a HICP protein” can be prepared by isolating a portion of SEQ ID NO: 1, SEQ ID NO:3 or SEQ ID NO: 12, which encodes a polypeptide having a HICP biological activity (the biological activities of the HICP proteins have previously been described), expressing the encoded portion of the HICP protein (e.g., by recombinant expression in vitro) and assessing the activity of the encoded portion of the HICP protein.

The invention further encompasses nucleic acid molecules that differ from the nucleotide sequence shown in SEQ ID NO: 1, SEQ ID NO:3 or SEQ ID NO: 12 due to degeneracy of the genetic code and thus encode the same HICP proteins as those encoded by the nucleotide sequence shown in SEQ ID NO: l, SEQ ID NO:3 or SEQ ID NO:12. In another embodiment, an isolated nucleic acid molecule of the invention has a nucleotide sequence encoding a protein having an amino acid sequence shown in SEQ ID NO:2 or SEQ ID NO: 13.

In addition to the rat HICP nucleotide sequences shown in SEQ ID NO:1, SEQ ID NO:3, or SEQ ID NO:12, it will be appreciated by those skilled in the art that DNA sequence polymorphisms that lead to changes in the amino acid sequences of the HICP proteins may exist within a population (e.g., the mouse population). Such genetic polymorphism in the HICP genes may exist among individuals within a population due to natural allelic variation. As used herein, the terms “gene” and “recombinant gene” refer to nucleic acid molecules comprising an open reading frame encoding a HICP protein, preferably a mammalian HICP protein. Such natural allelic variations can typically result in 1-5% variance in the nucleotide sequence of a HICP gene. Any and all such nucleotide variations and resulting amino acid polymorphisms in HICP genes that are the result of natural allelic variation and that do not alter the functional activity of a HICP protein are intended to be within the scope of the invention.

Moreover, nucleic acid molecules encoding HICP proteins from other species, and thus which have a nucleotide sequence which differs from the rat sequence of SEQ ID NO: 1, SEQ ID NO:3 or SEQ ID NO: 12 are intended to be within the scope of the invention. Nucleic acid molecules corresponding to natural allelic variants and homologues of the HICP cDNAs of the invention can be isolated based on their homology to the rat HICP nucleic acids disclosed herein using the rat cDNA, or a portion thereof, as a hybridization probe according to standard hybridization techniques under stringent hybridization conditions.

Accordingly, in another embodiment, an isolated nucleic acid molecule of the invention is at least 15 nucleotides in length and hybridizes under stringent conditions to the nucleic acid molecule comprising the nucleotide sequence of SEQ ID NO:1, SEQ ID NO:3, or SEQ ID NO:12. In other embodiment, the nucleic acid is at least 30, 50, 100, 250 or 500 nucleotides in length. As used herein, the term “hybridizes under stringent conditions” is intended to describe conditions for hybridization and washing under which nucleotide sequences at least 65% homologous to each other typically remain hybridized to each other. Preferably, the conditions are such that sequences at least about 70%, more preferably at least about 80%, even more preferably at least about 85% or 90% homologous to each other typically remain hybridized to each other. Such stringent conditions are known to those skilled in the art and can be found in Current Protocols in Molecular Biology, John Wiley & Sons, N.Y. (1989), 6.3.1-6.3.6. A preferred, non-limiting example of stringent hybridization conditions are hybridization in 6× sodium chloride/sodium citrate (SSC) at about 45° C., followed by one or more washes in 0.2× SSC, 0.1% SDS at 50-65° C. Preferably, an isolated nucleic acid molecule of the invention that hybridizes under stringent conditions to the sequence of SEQ ID NO:1, SEQ ID NO: 3 or SEQ ID NO;10 corresponds to a naturally-occurring nucleic acid molecule. As used herein, a “naturally-occurring” nucleic acid molecule refers to an RNA or DNA molecule having a nucleotide sequence that occurs in nature (e.g., encodes a natural protein).

In addition to naturally-occurring allelic variants of the HICP sequences that may exist in the population, the skilled artisan will further appreciate that changes can be introduced by mutation into the nucleotide sequences of SEQ ID NO:1, SEQ ID NO:3, or SEQ ID NO:12, thereby leading to changes in the amino acid sequence of the encoded HICP proteins, without altering the functional ability of the HICP proteins. For example, nucleotide substitutions leading to amino acid substitutions at “non-essential” amino acid residues can be made in the sequence of SEQ ID NO:1, SEQ ID NO:3, or SEQ ID NO:12. A “non-essential” amino acid residue is a residue that can be altered from the wild-type sequence of HICP (e.g., the sequence of SEQ ID NO:2) without altering the biological activity, whereas an “essential” amino acid residue is required for biological activity. For example, cysteine amino acid residues that are conserved among the HICP proteins of the present invention, are predicted to be particularly unamenable to alteration. Furthermore, amino acid residues that are conserved between HICP protein and other CCN protein family members are not likely to be amenable to alteration.

Accordingly, another aspect of the invention pertains to nucleic acid molecules encoding HICP proteins that contain changes in amino acid residues that are not essential for activity. Such HICP proteins differ in amino acid sequence from SEQ ID NO:2 yet retain biological activity. In one embodiment, the isolated nucleic acid molecule comprises a nucleotide sequence encoding a protein, wherein the protein comprises an amino acid sequence at least about 60% homologous to the amino acid sequence of SEQ ID NO:2 or SEQ ID NO:13. Preferably, the protein encoded by the nucleic acid molecule is at least about 65-70% homologous to SEQ ID NO:2 or SEQ ID NO:13, more preferably at least about 75-80% homologous to SEQ ID NO:2 or SEQ ID NO:13, even more preferably at least about 85-90% homologous to SEQ ID NO:2 or SEQ ID NO:13, and most preferably at least about 95% homologous to SEQ ID NO:2 or SEQ ID NO:13.

An isolated nucleic acid molecule encoding a HICP protein homologous to the protein of SEQ ID NO:2 or SEQ ID NO:13 can be created by introducing one or more nucleotide substitutions, additions or deletions into the nucleotide sequence of SEQ ID NO:1, SEQ ID NO:3 or SEQ ID NO:12, such that one or more amino acid substitutions, additions or deletions are introduced into the encoded protein. Mutations can be introduced into SEQ ID NO:1, SEQ ID NO:3 or SEQ ID NO:12 by standard techniques, such as site-directed mutagenesis and PCR-mediated mutagenesis. Preferably, conservative amino acid substitutions are made at one or more predicted non-essential amino acid residues. A “conservative amino acid substitution” is one in which the amino acid residue is replaced with an amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). Thus, a predicted nonessential amino acid residue in a HICP protein is preferably replaced with another amino acid residue from the same side chain family. Alternatively, in another embodiment, mutations can be introduced randomly along all or part of a HICP coding sequence, such as by saturation mutagenesis, and the resultant mutants can be screened for HICP biological activity to identify mutants that retain activity. Following mutagenesis of SEQ ID NO:1, SEQ ID NO:3 or SEQ ID NO:12, the encoded protein can be expressed recombinantly and the activity of the protein can be determined.

In a preferred embodiment, a mutant HICP protein can be assayed for the ability to modulate cellular proliferation, either in vitro or in vivo. In particular, a mutant HICP protein can be assayed for at least one of the following HICP activities: 1) it can modulate cell proliferation; 2) it can modulate a growth factor signaling pathway; 3) it can modulate the activity of CTGF or PDGF; 4) it can modulate a heparin-induced response in a heparin-responsive cell (e.g., a VSMC); 5) it can modulate cell motility; and 6) it can modulate extracellular proliferation.

In addition to the nucleic acid molecules encoding HICP proteins described above, another aspect of the invention pertains to isolated nucleic acid molecules which are antisense thereto. An “antisense” nucleic acid comprises a nucleotide sequence which is complementary to a “sense” nucleic acid encoding a protein, e.g., complementary to the coding strand of a double-stranded cDNA molecule or complementary to an mRNA sequence. Accordingly, an antisense nucleic acid can hydrogen bond to a sense nucleic acid. The antisense nucleic acid can be complementary to an entire HICP coding strand, or to only a portion thereof. In one embodiment, an antisense nucleic acid molecule is antisense to a “coding region” of the coding strand of a nucleotide sequence encoding HICP. The term “coding region” refers to the region of the nucleotide sequence comprising codons which are translated into amino acid residues (e.g., nucleotides 249 to 998 of SEQ ID NO:1, nucleotides 1-635 of SEQ ID NO:3 and nucleotides 1-612 of SEQ ID NO:12) Given the coding strand sequences encoding HICP disclosed herein (e.g., SEQ ID NO: 1, SEQ ID NO:3, SEQ ID NO: 12), antisense nucleic acids of the invention can be designed according to the rules of Watson and Crick base pairing. The antisense nucleic acid molecule can be complementary to the entire coding region of HICP mRNA, but more preferably is an oligonucleotide which is antisense to only a portion of the coding region of HICP mRNA. An antisense oligonucleotide can be, for example, about 5, 10, 15, 20, 25, 30, 35, 40, 45 or 50 nucleotides in length. An antisense nucleic acid of the invention can be constructed using chemical synthesis and enzymatic ligation reactions using procedures known in the art. For example, an antisense nucleic acid (e.g., an antisense oligonucleotide) can be chemically synthesized using naturally occurring nucleotides or variously modified nucleotides designed to increase the biological stability of the molecules or to increase the physical stability of the duplex formed between the antisense and sense nucleic acids, e.g., phosphorothioate derivatives and acridine substituted nucleotides can be used. Examples of modified nucleotides which can be used to generate the antisense nucleic acid include 5-fluorouracil, 5-bromouracil, 5-chlorouracil, 5-iodouracil, hypoxanthine, xantine, 4-acetylcytosine, 5-(carboxyhydroxylmethyl) uracil, 5-carboxymethylaminomethyl-2-thiouridine, 5-carboxymethylaminomethyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, beta-D-mannosylqueosine, 5′methoxycarboxymethyluracil, 5-methoxyuracil, 2-methylthio-N6-isopentenyladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5-oxyacetic acid methylester, uracil-5-oxyacetic acid (v), 5-methyl-2-thiouracil, 3-(3-amino-3-N-2-carboxypropyl) uracil, (acp3)w, and 2,6-diaminopurine. Alternatively, the antisense nucleic acid can be produced biologically using an expression vector into which a nucleic acid has been subcloned in an antisense orientation (i.e., RNA transcribed from the inserted nucleic acid will be of an antisense orientation to a target nucleic acid of interest, described further in the following subsection).

The antisense nucleic acid molecules of the invention are typically administered to a subject or generated in situ such that they hybridize with or bind to cellular mRNA and/or genomic DNA encoding a HICP protein to thereby inhibit expression of the protein, e.g., by inhibiting transcription and/or translation. The hybridization can be by conventional nucleotide complementarity to form a stable duplex, or, for example, in the case of an antisense nucleic acid molecule which binds to DNA duplexes, through specific interactions in the major groove of the double helix. An example of a route of administration of antisense nucleic acid molecules of the invention include direct injection at a tissue site. Alternatively, antisense nucleic acid molecules can be modified to target selected cells and then administered systemically. For example, for systemic administration, antisense molecules can be modified such that they specifically bind to receptors or antigens expressed on a selected cell surface, e.g., by linking the antisense nucleic acid molecules to peptides or antibodies which bind to cell surface receptors or antigens. The antisense nucleic acid molecules can also be delivered to cells using the vectors described herein. To achieve sufficient intracellular concentrations of the antisense molecules, vector constructs in which the antisense nucleic acid molecule is placed under the control of a strong pol II or pol III promoter are preferred.

In yet another embodiment, the antisense nucleic acid molecule of the invention is an α-anomeric nucleic acid molecule. An α-anomeric nucleic acid molecule forms specific double-stranded hybrids with complementary RNA in which, contrary to the usual β-units, the strands run parallel to each other (Gaultier et al. (1987) Nucleic Acids. Res. 15:6625-6641). The antisense nucleic acid molecule can also comprise a 2′-o-methylribonucleotide (Inoue et al. (1987) Nucleic Acids Res. 15:6131-6148) or a chimeric RNA-DNA analogue (Inoue et al. (1987) FEBS Lett. 215:327-330).

In still another embodiment, an antisense nucleic acid of the invention is a ribozyme. Ribozymes are catalytic RNA molecules with ribonuclease activity which are capable of cleaving a single-stranded nucleic acid, such as an mRNA, to which they have a complementary region. Thus, ribozymes (e.g., hammerhead ribozymes (described in Haselhoff and Gerlach (1988) Nature 334:585-591)) can be used to catalytically cleave HICP mRNA transcripts to thereby inhibit translation of HICP mRNA. A ribozyme having specificity for a HICP-encoding nucleic acid can be designed based upon the nucleotide sequence of a HICP cDNA disclosed herein (i.e., SEQ ID NO: 1, SEQ ID NO:3 or SEQ ID NO:12). For example, a derivative of a Tetrahymena L-19 IVS RNA can be constructed in which the nucleotide sequence of the active site is complementary to the nucleotide sequence to be cleaved in a HICP-encoding mRNA. See, e.g., Cech et al. U.S. Pat. No. 4,987,071; and Cech et al. U.S. Pat. No. 5,116,742. Alternatively, HICP mRNA can be used to select a catalytic RNA having a specific ribonuclease activity from a pool of RNA molecules. See, e.g., Bartel, D. and Szostak, J. W. (1993) Science 261:1411-1418.

Alternatively, HICP gene expression can be inhibited by targeting nucleotide sequences complementary to the regulatory region of the HICP (e.g., the HICP promoter and/or enhancers) to form triple helical structures that prevent transcription of the HICP gene in target cells. See generally, Helene, C. (1991) Anticancer Drug Des. 6(6):569-84; Helene, C. et al. (1992) Ann. N.Y. Acad. Sci. 660:27-36; and Maher, L. J. (1992) Bioassays 14(12):807-15.

In yet another embodiment, the HICP nucleic acid molecules of the present invention can be modified at the base moiety, sugar moiety or phosphate backbone to improve, e.g., the stability, hybridization, or solubility of the molecule. For example, the deoxyribose phosphate backbone of the nucleic acid molecules can be modified to generate peptide nucleic acids (see Hyrup B. et al. (1996) Bioorganic & Medicinal Chemistry 4 (1): 5-23). As used herein, the terms “peptide nucleic acids” or “PNAs” refer to nucleic acid mimics, e.g., DNA mimics, in which the deoxyribose phosphate backbone is replaced by a pseudopeptide backbone and only the four natural nucleobases are retained. The neutral backbone of PNAs has been shown to allow for specific hybridization to DNA and RNA under conditions of low ionic strength. The synthesis of PNA oligomers can be performed using standard solid phase peptide synthesis protocols as described in Hyrup B. et al. (1996) supra; Perry-O'Keefe et al. PNAS 93: 14670-675.

PNAs of HICP nucleic acid molecules can be used therapeutic and diagnostic applications. For example, PNAs can be used as antisense or antigene agents for sequence-specific modulation of gene expression by, for example, inducing transcription or translation arrest or inhibiting replication. PNAs of HICP nucleic acid molecules can also be used in the analysis of single base pair mutations in a gene, (e.g., by PNA-directed PCR clamping); as ‘artificial restriction enzymes’ when used in combination with other enzymes, (e.g., S1 nucleases (Hyrup B. (1996) supra)); or as probes or primers for DNA sequencing or hybridization (Hyrup B. et al. (1996) supra; Perry-O'Keefe supra).

In another embodiment, PNAs of HICP can be modified, (e.g., to enhance their stability or cellular uptake), by attaching lipophilic or other helper groups to PNA, by the formation of PNA-DNA chimeras, or by the use of liposomes or other techniques of drug delivery known in the art. For example, PNA-DNA chimeras of HICP nucleic acid molecules can be generated which may combine the advantageous properties of PNA and DNA. Such chimeras allow DNA recognition enzymes, (e.g., RNAse H and DNA polymerases), to interact with the DNA portion while the PNA portion would provide high binding affinity and specificity. PNA-DNA chimeras can be linked using linkers of appropriate lengths selected in terms of base stacking, number of bonds between the nucleobases, and orientation (Hyrup B. (1996) supra). The synthesis of PNA-DNA chimeras can be performed as described in Hyrup B. (1996) supra and Finn P.J. et al. (1996) Nucleic Acids Res. 24 (17): 3357-63. For example, a DNA chain can be synthesized on a solid support using standard phosphoramidite coupling chemistry and modified nucleoside analogs, e.g., 5′-(4-methoxytrityl)amino-5′-deoxy-thymidine phosphoramidite, can be used as a between the PNA and the 5′ end of DNA (Mag, M. et al. (1989) Nucleic Acid Res. 17: 5973-88). PNA monomers are then coupled in a stepwise manner to produce a chimeric molecule with a 5′ PNA segment and a 3′ DNA segment (Finn P.J. et al. (1996) supra). Alternatively, chimeric moleclues can be synthesized with a 5′ DNA segment and a 3′ PNA segment (Peterser, K. H. et al. (1975) Bioorganic Med. Chem. Lett. 5: 1119-11124).

In other embodiments, the oligonucleotide may include other appended groups such as peptides (e.g., for targeting host cell receptors in vivo), or agents facilitating transport across the cell membrane (see, e.g., Letsinger et al. (1989) Proc. Natl. Acad. Sci. US. 86:6553-6556; Lemaitre et al. (1987) Proc. Natl. Acad. Sci. USA 84:648-652; PCT Publication No. WO88/098 10, published Dec. 15, 1988) or the blood-brain barrier (see, e.g., PCT Publication No. WO89/10134, published Apr. 25, 1988). In addition, oligonucleotides can be modified with hybridization-triggered cleavage agents (See, e.g., Krol et al. (1988) Bio Techniques 6:958-976) or intercalating agents. (See, e.g., Zon (1988) Pharm. Res. 5:539-549). To this end, the oligonucleotide may be conjugated to another molecule, (e.g., a peptide, hybridization triggered cross-linking agent, transport agent, or hybridization-triggered cleavage agent).

II. Isolated HICP Proteins and Anti-HICP Antibodies

One aspect of the invention pertains to isolated HICP proteins, and biologically active portions thereof, as well as polypeptide fragments suitable for use as immunogens to raise anti-HICP antibodies. In one embodiment, native HICP proteins can be isolated from cells or tissue sources by an appropriate purification scheme using standard protein purification techniques. In another embodiment, HICP proteins are produced by recombinant DNA techniques. Alternative to recombinant expression, a HICP protein or polypeptide can be synthesized chemically using standard peptide synthesis techniques.

An “isolated” or “purified” protein or biologically active portion thereof is substantially free of cellular material or other contaminating proteins from the cell or tissue source from which the HICP protein is derived, or substantially free from chemical precursors or other chemicals when chemically synthesized. The language “substantially free of cellular material” includes preparations of HICP protein in which the protein is separated from cellular components of the cells from which it is isolated or recombinantly produced. In one embodiment, the language “substantially free of cellular material” includes preparations of HICP protein having less than about 30% (by dry weight) of non-HICP protein (also referred to herein as a “contaminating protein”), more preferably less than about 20% of non-HICP protein, still more preferably less than about 10% of non-HICP protein, and most preferably less than about 5% non-HICP protein. When the HICP protein or biologically active portion thereof is recombinantly produced, it is also preferably substantially free of culture medium, i.e., culture medium represents less than about 20%, more preferably less than about 10%, and most preferably less than about 5% of the volume of the protein preparation.

The language “substantially free of chemical precursors or other chemicals” includes preparations of HICP protein in which the protein is separated from chemical precursors or other chemicals which are involved in the synthesis of the protein. In one embodiment, the language “substantially free of chemical precursors or other chemicals” includes preparations of HICP protein having less than about 30% (by dry weight) of chemical precursors or non-HICP chemicals, more preferably less than about 20% chemical precursors or non-HICP chemicals, still more preferably less than about 10% chemical precursors or non-HICP chemicals, and most preferably less than about 5% chemical precursors or non-HICP chemicals.

Biologically active portions of a HICP protein include peptides comprising amino acid sequences sufficiently homologous to or derived from the amino acid sequence of the HICP protein, e.g., the amino acid sequence shown in SEQ ID NO:2 or SEQ ID NO:13, which include less amino acids than the full length HICP proteins, and exhibit at least one activity of a HICP protein. Typically, biologically active portions comprise a domain or motif with at least one activity of the HICP protein. A biologically active portion of a HICP protein can be a polypeptide which is, for example, 10, 25, 50, 100 or more amino acids in length.

In one embodiment, a biologically active portion of a HICP protein comprises at least a IGFBP motif, a VWC domain and/or a TSP1 motif. In yet another embodiment, a biologically active portion of a HICP protein comprises at least a signal sequence. In an alternative embodiment, a biologically active portion of a HICP protein comprises a HICP amino acid sequence lacking a signal sequence.

It is to be understood that a preferred biologically active portion of a HICP protein of the present invention may contain at least one of the above-identified structural domains. A more preferred biologically active portion of a HICP protein may contain at least two of the above-identified structural domains. An even more preferred biologically active portion of a HICP protein may contain at least three or more of the above-identified structural domains.

Moreover, other biologically active portions, in which other regions of the protein are deleted, can be prepared by recombinant techniques and evaluated for one or more of the functional activities of a native HICP protein.

In a preferred embodiment, the HICP protein has an amino acid sequence shown in SEQ ID NO:2 or SEQ ID NO:13. In other embodiments, the HICP protein is substantially homologous to SEQ ID NO:2 or SEQ ID NO:13 and retains the functional activity of the protein of SEQ ID NO:2 or SEQ ID NO:13 yet differs in amino acid sequence due to natural allelic variation or mutagenesis, as described in detail in subsection I above. Accordingly, in another embodiment, the HICP protein is a protein which comprises an amino acid sequence at least about 60% homologous to the amino acid sequence of SEQ ID NO:2 or SEQ ID NO:13 and retains the functional activity of the HICP proteins of SEQID NO:2 or SEQID NO:13. Preferably, the protein is at least about 70% homologous to SEQ ID NO:2 or SEQ ID NO:13, more preferably at least about 80% homologous to SEQ ID NO:2 or SEQ ID NO:13, even more preferably at least about 90% homologous to SEQ ID NO:2 or SEQ ID NO:13, and most preferably at least about 95% or more homologous to SEQ ID NO:2 or SEQ ID NO:13.

To determine the percent homology of two amino acid sequences or of two nucleic acids, the sequences are aligned for optimal comparison purposes (e.g., gaps can be introduced in the sequence of a first amino acid or nucleic acid sequence for optimal alignment with a second amino or nucleic acid sequence and non-homologous sequences can be disregarded for comparison purposes). In a preferred embodiment, the length of a reference sequence aligned for comparison purposes is at least 30%, preferably at least 40%, more preferably at least 50%, even more preferably at least 60%, and even more preferably at least 70%, 80%, or 90% of the length of the reference sequence (e.g., when aligning a second sequence to the HICP amino acid sequence of SEQ ID NO:2, having 86 amino acid residues, at least 26, preferably at least 46, more preferably at least 66 are aligned). The amino acid residues or nucleotides at corresponding amino acid positions or nucleotide positions are then compared. When a position in the first sequence is occupied by the same amino acid residue or nucleotide as the corresponding position in the second sequence, then the molecules are homologous at that position (i.e., as used herein amino acid or nucleic acid “homology” is equivalent to amino acid or nucleic acid “identity”). The percent homology between the two sequences is a function of the number of identical positions shared by the sequences (i.e., % homology=# of identical positions/total # of positions×100).

The comparison of sequences and determination of percent homology between two sequences can be accomplished using a mathematical algorithim. A preferred, non-limiting example of a mathematical algorithim utilized for the comparison of sequences is the algorithm of Karlin and Altschul (1990) Proc. Natl. Acad. Sci. USA 87:2264-68, modified as in Karlin and Altschul (1993) Proc. Natl. Acad. Sci. USA 90:5873-77. Such an algorithm is incorporated into the NBLAST and XBLAST programs (version 2.0) of Altschul, et al. (1990) J. Mol. Biol. 215:403-10. BLAST nucleotide searches can be performed with the NBLAST program, score=100, wordlength=12 to obtain nucleotide sequences homologous to HICP nucleic acid molecules of the invention. BLAST protein searches can be performed with the XBLAST program, score=50, wordlength=3 to obtain amino acid sequences homologous to TAP-1 protein molecules of the invention. To obtain gapped alignments for comparison purposes, Gapped BLAST can be utilized as described in Altschul et al., (1997) Nucleic Acids Research 25(17):3389-3402. When utilizing BLAST and Gapped BLAST programs, the default parameters of the respective programs (e.g., XBLAST and NBLAST) can be used. See http://www.ncbi.nlm.nih.gov. Another preferred, non-limiting example of a mathematical algorithim utilized for the comparison of sequences is the algorithm of Myers and Miller, CABIOS (1989). Such an algorithm is incorporated into the ALIGN program (version 2.0) which is part of the GCG sequence alignment software package. When utilizing the ALIGN program for comparing amino acid sequences, a PAM120 weight residue table, a gap length penalty of 12, and a gap penalty of 4 can be used.

The invention also provides HICP chimeric or fusion proteins. As used herein, a HICP “chimeric protein” or “fusion protein” comprises a HICP polypeptide operatively linked to a non-HICP polypeptide. A “HICP polypeptide” refers to a polypeptide having an amino acid sequence corresponding to HICP, whereas a “non-HICP polypeptide” refers to a polypeptide having an amino acid sequence corresponding to a protein which is not substantially homologous to the HICP protein, e.g., a protein which is different from the HICP protein and which is derived from the same or a different organism. Within a HICP fusion protein the HICP polypeptide can correspond to all or a portion of a HICP protein. In a preferred embodiment, a HICP fusion protein comprises at least one biologically active portion of a HICP protein. In another preferred embodiment, a HICP fusion protein comprises at least two biologically active portions of a HICP protein. In another preferred embodiment, a HICP fusion protein comprises at least three biologically active portions of a HICP protein. Within the fusion protein, the term “operatively linked” is intended to indicate that the HICP polypeptide and the non-HICP polypeptide are fused in-frame to each other. The non-HICP polypeptide can be fused to the N-terminus or C-terminus of the HICP polypeptide.

For example, in one embodiment, the fusion protein is a GST-HICP fusion protein in which the HICP sequences are fused to the C-terminus of the GST sequences. Such fusion proteins can facilitate the purification of recombinant HICP.

In another embodiment, the fusion protein is a HICP protein containing a heterologous signal sequence at its N-terminus. For example, the native HICP signal sequence (i.e, about amino acids 1 to 23 of SEQ ID NO:2) can be removed and replaced with a signal sequence from another protein. In certain host cells (e.g., mammalian host cells), expression and/or secretion of HICP can be increased through use of a heterologous signal sequence.

In yet another embodiment, the fusion protein is a HICP-immunoglobulin fusion protein in which the HICP sequences comprising primarily the HICP extracellular domain are fused to sequences derived from a member of the immunoglobulin protein family. Soluble derivatives have also been made of cell surface glycoproteins in the immunoglobulin gene superfamily consisting of an extracellular domain of the cell surface glycoprotein fused to an immunoglobulin constant (Fc) region (see e.g., Capon, D. J. et al. (1989) Nature 337:525-531 and Capon U.S. Pat. Nos. 5,116,964 and 5,428,130 [CD4-IgG1 constructs]; Linsley, P. S. et al. (1991) J. Exp. Med. 173:721-730 [a CD28-IgG1 construct and a B7-1-IgG1 construct]; and Linsley, P. S. et al. (1991) J. Exp. Med. 174:561-569 and U.S. Pat. No. 5,434,131[a CTLA4-IgG1]). Such fusion proteins have proven useful for modulating receptor-ligand interactions. Soluble derivatives of cell surface proteins of the tumor necrosis factor receptor (TNFR) superfamily proteins have been made consisting of an extracellular domain of the cell surface receptor fused to an immunoglobulin constant (Fc) region (see for example Moreland et al. (1997) N. Engl. J. Med. 337(3):141-147; van der Poll et al. (1997) Blood 89(10):3727-3734; and Ammann et al. (1997) J. Clin. Invest. 99(7):1699-1703.) Furthermore, fusion proteins have been made using the CH2 and CH3 domains of IgG fused downstream of murine IL-17 leader sequences and upstream of murine CTLA-8 sequences and upstream of HVS13 sequences (see for example Yao et al. (1995) Immunity 8:811-821.)

The HICP-immunoglobulin fusion proteins of the invention can be incorporated into pharmaceutical compositions and administered to a subject to inhibit an interaction between a HICP protein and a HICP receptor on the surface of a cell, to thereby suppress HICP-mediated cellular function in vivo. The HICP-immunoglobulin fusion proteins can be used to affect the bioavailability of a HICP protein. Inhibition of the HICP protein/HICP receptor interaction may be useful therapeutically, for example, in regulation of the cellular proliferation. Moreover, the HICP-immunoglobulin fusion proteins of the invention can be used as immunogens to produce anti-HICP antibodies in a subject, to purify HICP receptors and in screening assays to identify molecules which inhibit the interaction of a HICP protein with a HICP receptor.

Preferably, a HICP chimeric or fusion protein of the invention is produced by standard recombinant DNA techniques. For example, DNA fragments coding for the different polypeptide sequences are ligated together in-frame in accordance with conventional techniques, for example by employing blunt-ended or stagger-ended termini for ligation, restriction enzyme digestion to provide for appropriate termini, filling-in of cohesive ends as appropriate, alkaline phosphatase treatment to avoid undesirable joining, and enzymatic ligation. In another embodiment, the fusion gene can be synthesized by conventional techniques including automated DNA synthesizers. Alternatively, PCR amplification of gene fragments can be carried out using anchor primers which give rise to complementary overhangs between two consecutive gene fragments which can subsequently be annealed and reamplified to generate a chimeric gene sequence (see, for example, Current Protocols in Molecular Biology, eds. Ausubel et al. John Wiley & Sons: 1992). Moreover, many expression vectors are commercially available that already encode a fusion moiety (e.g., a GST polypeptide). A HICP-encoding nucleic acid can be cloned into such an expression vector such that the fusion moiety is linked in-frame to the HICP protein.

The present invention also pertains to variants of the HICP proteins which function as either HICP agonists (mimetics) or as HICP antagonists. Variants of the HICP protein can be generated by mutagenesis, e.g., discrete point mutation or truncation of the HICP protein. An agonist of the HICP protein can retain substantially the same, or a subset, of the biological activities of the naturally occurring form of the HICP protein. An antagonist of the HICP protein can inhibit one or more of the activities of the naturally occurring form of the HICP protein by, for example, competitively binding to a HICP receptor of HICP-binding protein. Thus, specific biological effects can be elicited by treatment with a variant of limited function. In one embodiment, treatment of a subject with a variant having a subset of the biological activities of the naturally occurring form of the protein has fewer side effects in a subject relative to treatment with the naturally occurring form of the HICP proteins.

In one embodiment, variants of the HICP protein which function as either HICP agonists (mimetics) or as HICP antagonists can be identified by screening combinatorial libraries of mutants, (e.g., truncation mutants) of the HICP protein for HICP protein agonist or antagonist activity. In one embodiment, a variegated library of HICP variants is generated by combinatorial mutagenesis at the nucleic acid level and is encoded by a variegated gene library. A variegated library of HICP variants can be produced by, for example, enzymatically ligating a mixture of synthetic oligonucleotides into gene sequences such that a degenerate set of potential HICP sequences is expressible as individual polypeptides, or alternatively, as a set of larger fusion proteins (e.g., for phage display) containing the set of HICP sequences therein. There are a variety of methods which can be used to produce libraries of potential HICP variants from a degenerate oligonucleotide sequence. Chemical synthesis of a degenerate gene sequence can be performed in an automatic DNA synthesizer, and the synthetic gene then ligated into an appropriate expression vector. Use of a degenerate set of genes allows for the provision, in one mixture, of all of the sequences encoding the desired set of potential HICP sequences. Methods for synthesizing degenerate oligonucleotides are known in the art (see, e.g., Narang, S. A. (1983) Tetrahedron 39:3; Itakura et al. (1984) Annu. Rev. Biochem. 53:323; Itakura et al. (1984) Science 198:1056; Ike et al. (1983) Nucleic Acid Res. 11:477.

In addition, libraries of fragments of the HICP protein coding sequence can be used to generate a variegated population of HICP fragments for screening and subsequent selection of variants of a HICP protein. In one embodiment, a library of coding sequence fragments can be generated by treating a double stranded PCR fragment of a HICP coding sequence with a nuclease under conditions wherein nicking occurs only about once per molecule, denaturing the double stranded DNA, renaturing the DNA to form double stranded DNA which can include sense/antisense pairs from different nicked products, removing single stranded portions from reformed duplexes by treatment with SI nuclease, and ligating the resulting fragment library into an expression vector. By this method, an expression library can be derived which encodes N-terminal, C-terminal and internal fragments of various sizes of the HICP protein.

Several techniques are known in the art for screening gene products of combinatorial libraries made by point mutations or truncation, and for screening cDNA libraries for gene products having a selected property. Such techniques are adaptable for rapid screening of the gene libraries generated by the combinatorial mutagenesis of HICP proteins. The most widely used techniques, which are amenable to high through-put analysis, for screening large gene libraries typically include cloning the gene library into replicable expression vectors, transforming appropriate cells with the resulting library of vectors, and expressing the combinatorial genes under conditions in which detection of a desired activity facilitates isolation of the vector encoding the gene whose product was detected. Recrusive ensemble mutagenesis (REM), a new technique which enhances the frequency of functional mutants in the libraries, can be used in combination with the screening assays to identify HICP variants (Arkin and Yourvan (1992) PNAS 89:7811-7815; Delgrave et al. (1993) Protein Engineering 6(3):327-331).

In one embodiment, cell based assays can be exploited to analyze a variegated HICP library. For example, a library of expression vectors can be transfected into a cell line which ordinarily secretes HICP protein. Supernatants from the transfected cells are then contacted with HICP-responsive cells and the effect of the mutation in HICP can be detected, e.g., by measuring any of a number of HICP-responsive cell responses. Plasmid DNA can then be recovered from the mutant HICP-secreting cells which score for inhibition, or alternatively, potentiation of the HICP-dependent response, and the individual clones further characterized.

An isolated HICP protein, or a portion or fragment thereof, can be used as an immunogen to generate antibodies that bind HICP using standard techniques for polyclonal and monoclonal antibody preparation. The full-length HICP protein can be used or, alternatively, the invention provides antigenic peptide fragments of HICP for use as immunogens. The antigenic peptide of HICP comprises at least 8 amino acid residues of the amino acid sequence shown in SEQ ID NO:2 or SEQ ID NO:13 and encompasses an epitope of HICP such that an antibody raised against the peptide forms a specific immune complex with HICP. Preferably, the antigenic peptide comprises at least 10 amino acid residues, more preferably at least 15 amino acid residues, even more preferably at least 20 amino acid residues, and most preferably at least 30 amino acid residues.

A HICP immunogen typically is used to prepare antibodies by immunizing a suitable subject, (e.g., rabbit, goat, mouse or other mammal) with the immunogen. An appropriate immunogenic preparation can contain, for example, recombinantly expressed HICP protein or a chemically synthesized HICP polypeptide. The preparation can further include an adjuvant, such as Freund's complete or incomplete adjuvant, or similar immunostimulatory agent. Immunization of a suitable subject with an immunogenic HICP preparation induces a polyclonal anti-HICP antibody response.

Accordingly, another aspect of the invention pertains to anti-HICP antibodies. The term “antibody” as used herein refers to immunoglobulin molecules and immunologically active portions of immunoglobulin molecules, i.e., molecules that contain an antigen binding site which specifically binds (immunoreacts with) an antigen, such as HICP. Examples of immunologically active portions of immunoglobulin molecules include F(ab) and F(ab′)₂ fragments which can be generated by treating the antibody with an enzyme such as pepsin. The invention provides polyclonal and monoclonal antibodies that bind HICP. The term “monoclonal antibody” or “monoclonal antibody composition”, as used herein, refers to a population of antibody molecules that contain only one species of an antigen binding site capable of immunoreacting with a particular epitope of HICP. A monoclonal antibody composition thus typically displays a single binding affinity for a particular HICP protein with which it immunoreacts.

Polyclonal anti-HICP antibodies can be prepared as described above by immunizing a suitable subject with a HICP immunogen. The anti-HICP antibody titer in the immunized subject can be monitored over time by standard techniques, such as with an enzyme linked immunosorbent assay (ELISA) using immobilized HICP. If desired, the antibody molecules directed against HICP can be isolated from the mammal (e.g., from the blood) and further purified by well known techniques, such as protein A chromatography to obtain the IgG fraction. At an appropriate time after immunization, e.g., when the anti-HICP antibody titers are highest, antibody-producing cells can be obtained from the subject and used to prepare monoclonal antibodies by standard techniques, such as the hybridoma technique originally described by Kohler and Milstein (1975) Nature 256:495-497) (see also, Brown et al. (1981) J. Immunol. 127:539-46; Brown et al. (1980) J. Biol. Chem .255:4980-83; Yeh et al. (1976) PNAS 76:2927-31; and Yeh et al. (1982) Int. J. Cancer 29:269-75), the more recent human B cell hybridoma technique (Kozbor et al. (1983) Immunol Today 4:72), the EBV-hybridoma technique (Cole et al. (1985), Monoclonal Antibodies and Cancer Therapy, Alan R. Liss, Inc., pp. 77-96) or trioma techniques. The technology for producing monoclonal antibody hybridomas is well known (see generally R. H. Kenneth, in Monoclonal Antibodies: A New Dimension In Biological Analyses, Plenum Publishing Corp., New York, N.Y. (1980); E. A. Lerner (1981) Yale J. Biol. Med., 54:387-402; M. L. Gefter et al. (1977) Somatic Cell Genet. 3:231-36). Briefly, an immortal cell line (typically a myeloma) is fused to lymphocytes (typically splenocytes) from a mammal immunized with a HICP immunogen as described above, and the culture supernatants of the resulting hybridoma cells are screened to identify a hybridoma producing a monoclonal antibody that binds HICP.

Any of the many well known protocols used for fusing lymphocytes and immortalized cell lines can be applied for the purpose of generating an anti-HICP monoclonal antibody (see, e.g., G. Galfre et al. (1977) Nature 266:55052; Gefter et al. Somatic Cell Genet., cited supra; Lerner, Yale J. Biol. Med., cited supra; Kenneth, Monoclonal Antibodies, cited supra). Moreover, the ordinarily skilled worker will appreciate that there are many variations of such methods which also would be useful. Typically, the immortal cell line (e.g., a myeloma cell line) is derived from the same mammalian species as the lymphocytes. For example, murine hybridomas can be made by fusing lymphocytes from a mouse immunized with an immunogenic preparation of the present invention with an immortalized mouse cell line. Preferred immortal cell lines are mouse myeloma cell lines that are sensitive to culture medium containing hypoxanthine, aminopterin and thymidine (“HAT medium”). Any of a number of myeloma cell lines can be used as a fusion partner according to standard techniques, e.g., the P3-NS1/1-Ag4-1, P3-x63-Ag8.653 or Sp2/O-Ag14 myeloma lines. These myeloma lines are available from ATCC. Typically, HAT-sensitive mouse myeloma cells are fused to mouse splenocytes using polyethylene glycol (“PEG”). Hybridoma cells resulting from the fusion are then selected using HAT medium, which kills unfused and unproductively fused myeloma cells (unfused splenocytes die after several days because they are not transformed). Hybridoma cells producing a monoclonal antibody of the invention are detected by screening the hybridoma culture supernatants for antibodies that bind HICP, e.g., using a standard ELISA assay.

Alternative to preparing monoclonal antibody-secreting hybridomas, a monoclonal anti-HICP antibody can be identified and isolated by screening a recombinant combinatorial immunoglobulin library (e.g., an antibody phage display library) with HICP to thereby isolate immunoglobulin library members that bind HICP. Kits for generating and screening phage display libraries are commercially available (e.g., the Pharmacia Recombinant Phage Antibody System, Catalog No. 27-9400-01; and the Stratagene SurfZAP™ Phage Display Kit, Catalog No. 240612). Additionally, examples of methods and reagents particularly amenable for use in generating and screening antibody display library can be found in, for example, Ladner et al. U.S. Pat. No. 5,223,409; Kang et al. PCT International Publication No. WO 92/18619; Dower et al. PCT International Publication No. WO 91/17271; Winter et al. PCT International Publication WO 92/20791; Markland et al. PCT International Publication No. WO 92/15679; Breitling et al. PCT International Publication WO 93/01288; McCafferty et al. PCT International Publication No. WO 92/01047; Garrard et al. PCT International Publication No. WO 92/09690; Ladner et al. PCT International Publication No. WO 90/02809; Fuchs et al. (1991) Bio/Technology 9:1370-1372; Hay et al. (1992) Hum. Antibod. Hybridomas 3:81-85; Huse et al. (1989) Science 246:1275-1281; Griffiths et al. (1993) EMBO J 12:725-734; Hawkins et al. (1992) J. Mol. Biol. 226:889-896; Clarkson et al. (1991) Nature 352:624-628; Gram et al. (1992) PNAS 89:3576-3580; Garrad et al. (1991) Bio/Technology 9:1373-1377; Hoogenboom et al. (1991) Nuc. Acid Res. 19:4133-4137; Barbas et al. (1991) PNAS 88:7978-7982; and McCafferty et al. Nature (1990) 348:552-554.

Additionally, recombinant anti-HICP antibodies, such as chimeric and humanized monoclonal antibodies, comprising both human and non-human portions, which can be made using standard recombinant DNA techniques, are within the scope of the invention. Such chimeric and humanized monoclonal antibodies can be produced by recombinant DNA techniques known in the art, for example using methods described in Robinson et al. International Application No. PCT/US86/02269; Akira, et al. European Patent Application 184,187; Taniguchi, M., European Patent Application 171,496; Morrison et al. European Patent Application 173,494; Neuberger et al. PCT International Publication No. WO 86/01533; Cabilly et al. U.S. Pat. No. 4,816,567; Cabilly et al. European Patent Application 125,023; Better et al. (1988) Science 240:1041-1043; Liu et al. (1987) PNAS 84:3439-3443; Liu et al. (1987) J. Immunol. 139:3521-3526; Sun et al. (1987) PNAS 84:214-218; Nishimura et al. (1987) Canc. Res. 47:999-1005; Wood et al. (1985) Nature 314:446-449; and Shaw et al. (1988) J. Natl. Cancer Inst. 80:1553-1559); Morrison, S. L. (1985) Science 229:1202-1207; Oi et al. (1986) BioTechniques 4:214; Winter U.S. Pat. No. 5,225,539; Jones et al. (1986) Nature 321:552-525; Verhoeyan et al. (1988) Science 239:1534; and Beidler et al. (1988) J. Immunol. 141:4053-4060.

An anti-HICP antibody (e.g., monoclonal antibody) can be used to isolate HICP by standard techniques, such as affinity chromatography or immunoprecipitation. An anti-HICP antibody can facilitate the purification of natural HICP from cells and of recombinantly produced HICP expressed in host cells. Moreover, an anti-HICP antibody can be used to detect HICP protein (e.g., in a cellular lysate or cell supernatant) in order to evaluate the abundance and pattern of expression of the HICP protein. Anti-HICP antibodies can be used diagnostically to monitor protein levels in tissue as part of a clinical testing procedure, e.g., to, for example, determine the efficacy of a given treatment regimen. Detection can be facilitated by coupling (i.e., physically linking) the antibody to a detectable substance. Examples of detectable substances include various enzymes, prosthetic groups, fluorescent materials, luminescent materials, bioluminescent materials, and radioactive materials. Examples of suitable enzymes include horseradish peroxidase, alkaline phosphatase, β-galactosidase, or acetylcholinesterase; examples of suitable prosthetic group complexes include streptavidin/biotin and avidin/biotin; examples of suitable fluorescent materials include umbelliferone, fluorescein, fluorescein isothiocyanate, rhodamine, dichlorotriazinylamine fluorescein, dansyl chloride or phycoerythrin; an example of a luminescent material includes luminol; examples of bioluminescent materials include luciferase, luciferin, and aequorin, and examples of suitable radioactive material include ¹²⁵I, ¹³¹ I, ³⁵S or ³H.

III. Recombinant Expression Vectors and Host Cells

Another aspect of the invention pertains to vectors, preferably expression vectors, containing a nucleic acid encoding HICP (or a portion thereof). As used herein, the term “vector” refers to a nucleic acid molecule capable of transporting another nucleic acid to which it has been linked. One type of vector is a “plasmid”, which refers to a circular double stranded DNA loop into which additional DNA segments can be ligated. Another type of vector is a viral vector, wherein additional DNA segments can be ligated into the viral genome. Certain vectors are capable of autonomous replication in a host cell into which they are introduced (e.g., bacterial vectors having a bacterial origin of replication and episomal mammalian vectors). Other vectors (e.g., non-episomal mammalian vectors) are integrated into the genome of a host cell upon introduction into the host cell, and thereby are replicated along with the host genome. Moreover, certain vectors are capable of directing the expression of genes to which they are operatively linked. Such vectors are referred to herein as “expression vectors”. In general, expression vectors of utility in recombinant DNA techniques are often in the form of plasmids. In the present specification, “plasmid” and “vector” can be used interchangeably as the plasmid is the most commonly used form of vector. However, the invention is intended to include such other forms of expression vectors, such as viral vectors (e.g., replication defective retroviruses, adenoviruses and adeno-associated viruses), which serve equivalent functions.

The recombinant expression vectors of the invention comprise a nucleic acid of the invention in a form suitable for expression of the nucleic acid in a host cell, which means that the recombinant expression vectors include one or more regulatory sequences, selected on the basis of the host cells to be used for expression, which is operatively linked to the nucleic acid sequence to be expressed. Within a recombinant expression vector, “operably linked” is intended to mean that the nucleotide sequence of interest is linked to the regulatory sequence(s) in a manner which allows for expression of the nucleotide sequence (e.g., in an in vitro transcription/translation system or in a host cell when the vector is introduced into the host cell). The term “regulatory sequence” is intended to includes promoters, enhancers and other expression control elements (e.g., polyadenylation signals). Such regulatory sequences are described, for example, in Goeddel; Gene Expression Technology: Methods in Enzymology 185, Academic Press, San Diego, Calif. (1990). Regulatory sequences include those which direct constitutive expression of a nucleotide sequence in many types of host cell and those which direct expression of the nucleotide sequence only in certain host cells (e.g., tissue-specific regulatory sequences). It will be appreciated by those skilled in the art that the design of the expression vector can depend on such factors as the choice of the host cell to be transformed, the level of expression of protein desired, etc. The expression vectors of the invention can be introduced into host cells to thereby produce proteins or peptides, including fusion proteins or peptides, encoded by nucleic acids as described herein (e.g., HICP proteins, mutant forms of HICP, fusion proteins, etc.).

The recombinant expression vectors of the invention can be designed for expression of HICP in prokaryotic or eukaryotic cells. For example, HICP can be expressed in bacterial cells such as E. coli, insect cells (using baculovirus expression vectors) yeast cells or mammalian cells. Suitable host cells are discussed further in Goeddel, Gene Expression Technology: Methods in Enzymology 185, Academic Press, San Diego, Calif. (1990). Alternatively, the recombinant expression vector can be transcribed and translated in vitro, for example using T7 promoter regulatory sequences and T7 polymerase.

Expression of proteins in prokaryotes is most often carried out in E. coli with vectors containing constitutive or inducible promoters directing the expression of either fusion or non-fusion proteins. Fusion vectors add a number of amino acids to a protein encoded therein, usually to the amino terminus of the recombinant protein. Such fusion vectors typically serve three purposes: 1) to increase expression of recombinant protein; 2) to increase the solubility of the recombinant protein; and 3) to aid in the purification of the recombinant protein by acting as a ligand in affinity purification. Often, in fusion expression vectors, a proteolytic cleavage site is introduced at the junction of the fusion moiety and the recombinant protein to enable separation of the recombinant protein from the fusion moiety subsequent to purification of the fusion protein. Such enzymes, and their cognate recognition sequences, include Factor Xa, thrombin and enterokinase. Typical fusion expression vectors include pGEX (Pharmacia Biotech Inc; Smith, D. B. and Johnson, K. S. (1988) Gene 67:31-40), pMAL (New England Biolabs, Beverly, Mass.) and pRIT5 (Pharmacia, Piscataway, N.J.) which fuse glutathione S-transferase (GST), maltose E binding protein, or protein A, respectively, to the target recombinant protein.

Purified fusion proteins can be utilized in HICP activity assays, in HICP ligand binding (e.g., direct assays or competitive assays described in detail below), to generate antibodies specific for HICP proteins, as examples.

Examples of suitable inducible non-fusion E. coli expression vectors include pTrc (Amann et al., (1988) Gene 69:301-315) and pET 11d (Studier et al., Gene Expression Technology: Methods in Enzymology 185, Academic Press, San Diego, Calif. (1990) 60-89). Target gene expression from the pTrc vector relies on host RNA polymerase transcription from a hybrid trp-lac fusion promoter. Target gene expression from the pET 11d vector relies on transcription from a T7 gn10-lac fusion promoter mediated by a coexpressed viral RNA polymerase (T7 gn1). This viral polymerase is supplied by host strains BL21(DE3) or HMS174(DE3) from a resident λ prophage harboring a T7 gn1 gene under the transcriptional control of the lacUV 5 promoter.

One strategy to maximize recombinant protein expression in E. coli is to express the protein in a host bacteria with an impaired capacity to proteolytically cleave the recombinant protein (Gottesman, S., Gene Expression Technology: Methods in Enzymology 185, Academic Press, San Diego, Calif. (1990) 119-128). Another strategy is to alter the nucleic acid sequence of the nucleic acid to be inserted into an expression vector so that the individual codons for each amino acid are those preferentially utilized in E. coli (Wada et al., (1992) Nucleic Acids Res. 20:2111-2118). Such alteration of nucleic acid sequences of the invention can be carried out by standard DNA synthesis techniques.

In another embodiment, the HICP expression vector is a yeast expression vector. Examples of vectors for expression in yeast S. cerivisae include pYepSec1 (Baldari, et al., (1987) Embo J 6:229-234), pMFa (Kurjan and Herskowitz, (1982) Cell 30:933-943), pJRY88 (Schultz et al., (1987) Gene 54:113-123), pYES2 (Invitrogen Corporation, San Diego, Calif.), and picZ (InVitrogen Corp, San Diego, Calif.).

Alternatively, HICP can be expressed in insect cells using baculovirus expression vectors. Baculovirus vectors available for expression of proteins in cultured insect cells (e.g., Sf 9 cells) include the pAc series (Smith et al. (1983) Mol. Cell Biol. 3:2156-2165) and the pVL series (Lucklow and Summers (1989) Virology 170:31-39).

In yet another embodiment, a nucleic acid of the invention is expressed in mammalian cells using a mammalian expression vector. Examples of mammalian expression vectors include pCDM8 (Seed, B. (1987) Nature 329:840) and pMT2PC (Kaufman et al. (1987) EMBO J 6:187-195). When used in mammalian cells, the expression vector's control functions are often provided by viral regulatory elements. For example, commonly used promoters are derived from polyoma, Adenovirus 2, cytomegalovirus and Simian Virus 40. For other suitable expression systems for both prokaryotic and eukaryotic cells see chapters 16 and 17 of Sambrook, J., Fritsh, E. F., and Maniatis, T. Molecular Cloning: A Laboratory Manual. 2nd, ed., Cold Spring Harbor Laboratory, Cold Spring Harbor Laboratory Press, Cold Spring Harbor, N.Y., 1989.

In another embodiment, the recombinant mammalian expression vector is capable of directing expression of the nucleic acid preferentially in a particular cell type (e.g., tissue-specific regulatory elements are used to express the nucleic acid). Tissue-specific regulatory elements are known in the art. Non-limiting examples of suitable tissue-specific promoters include the albumin promoter (liver-specific; Pinkert et al. (1987) Genes Dev. 1:268-277), lymphoid-specific promoters (Calame and Eaton (1988) Adv. Immunol. 43:235-275), in particular promoters of T cell receptors (Winoto and Baltimore (1989) EMBO J. 8:729-733) and immunoglobulins (Banerji et al. (1983) Cell 33:729-740; Queen and Baltimore (1983) Cell 33:741-748), neuron-specific promoters (e.g., the neurofilament promoter; Byrne and Ruddle (1989) PNAS 86:5473-5477), pancreas-specific promoters (Edlund et al. (1985) Science 230:912-916), and mammary gland-specific promoters (e.g., milk whey promoter; U.S. Pat. No. 4,873,316 and European Application Publication No. 264,166). Developmentally-regulated promoters are also encompassed, for example the murine hox promoters (Kessel and Gruss (1990) Science 249:374-379) and the α-fetoprotein promoter (Campes and Tiilghman (1989) Genes Dev. 3:537-546).

The invention further provides a recombinant expression vector comprising a DNA molecule of the invention cloned into the expression vector in an antisense orientation. That is, the DNA molecule is operatively linked to a regulatory sequence in a manner which allows for expression (by transcription of the DNA molecule) of an RNA molecule which is antisense to HICP mRNA. Regulatory sequences operatively linked to a nucleic acid cloned in the antisense orientation can be chosen which direct the continuous expression of the antisense RNA molecule in a variety of cell types, for instance viral promoters and/or enhancers, or regulatory sequences can be chosen which direct constitutive, tissue specific or cell type specific expression of antisense RNA. The antisense expression vector can be in the form of a recombinant plasmid, phagemid or attenuated virus in which antisense nucleic acids are produced under the control of a high efficiency regulatory region, the activity of which can be determined by the cell type into which the vector is introduced. For a discussion of the regulation of gene expression using antisense genes see Weintraub, H. et al., Antisense RNA as a molecular tool for genetic analysis, Reviews—Trends in Genetics, Vol. 1(1) 1986.

Another aspect of the invention pertains to host cells into which a recombinant expression vector of the invention has been introduced. The terms “host cell” and “recombinant host cell” are used interchangeably herein. It is understood that such terms refer not only to the particular subject cell but to the progeny or potential progeny of such a cell. Because certain modifications may occur in succeeding generations due to either mutation or environmental influences, such progeny may not, in fact, be identical to the parent cell, but are still included within the scope of the term as used herein.

A host cell can be any prokaryotic or eukaryotic cell. For example, HICP protein can be expressed in bacterial cells such as E. coli, insect cells, yeast or mammalian cells (such as Chinese hamster ovary cells (CHO) or COS cells). Other suitable host cells are known to those skilled in the art.

Vector DNA can be introduced into prokaryotic or eukaryotic cells via conventional transformation or transfection techniques. As used herein, the terms “transformation” and “transfection” are intended to refer to a variety of art-recognized techniques for introducing foreign nucleic acid (e.g., DNA) into a host cell, including calcium phosphate or calcium chloride co-precipitation, DEAE-dextran-mediated transfection, lipofection, or electroporation. Suitable methods for transforming or transfecting host cells can be found in Sambrook, et al. (Molecular Cloning: A Laboratory Manual. 2nd, ed., Cold Spring Harbor Laboratory, Cold Spring Harbor Laboratory Press, Cold Spring Harbor, N.Y., 1989), and other laboratory manuals.

For stable transfection of mammalian cells, it is known that, depending upon the expression vector and transfection technique used, only a small fraction of cells may integrate the foreign DNA into their genome. In order to identify and select these integrants, a gene that encodes a selectable marker (e.g., resistance to antibiotics) is generally introduced into the host cells along with the gene of interest. Preferred selectable markers include those which confer resistance to drugs, such as G418, hygromycin and methotrexate. Nucleic acid encoding a selectable marker can be introduced into a host cell on the same vector as that encoding HICP or can be introduced on a separate vector. Cells stably transfected with the introduced nucleic acid can be identified by drug selection (e.g., cells that have incorporated the selectable marker gene will survive, while the other cells die).

A host cell of the invention, such as a prokaryotic or eukaryotic host cell in culture, can be used to produce (i.e., express) HICP protein. Accordingly, the invention further provides methods for producing HICP protein using the host cells of the invention. In one embodiment, the method comprises culturing the host cell of invention (into which a recombinant expression vector encoding HICP has been introduced) in a suitable medium such that HICP protein is produced. In another embodiment, the method further comprises isolating HICP from the medium or the host cell.

The host cells of the invention can also be used to produce nonhuman transgenic animals. For example, in one embodiment, a host cell of the invention is a fertilized oocyte or an embryonic stem cell into which HICP-coding sequences have been introduced. Such host cells can then be used to create non-human transgenic animals in which exogenous HICP sequences have been introduced into their genome or homologous recombinant animals in which endogenous HICP sequences have been altered. Such animals are useful for studying the function and/or activity of HICP and for identifying and/or evaluating modulators of HICP activity. As used herein, a “transgenic animal” is a non-human animal, preferably a mammal, more preferably a rodent such as a mouse, in which one or more of the cells of the animal includes a transgene. Other examples of transgenic animals include non-human primates, sheep, dogs, cows, goats, chickens, amphibians, etc. A transgene is exogenous DNA which is integrated into the genome of a cell from which a transgenic animal develops and which remains in the genome of the mature animal, thereby directing the expression of an encoded gene product in one or more cell types or tissues of the transgenic animal. As used herein, a “homologous recombinant animal” is a non-human animal, preferably a mammal, more preferably a mouse, in which an endogenous HICP gene has been altered by homologous recombination between the endogenous gene and an exogenous DNA molecule introduced into a cell of the animal, e.g., an embryonic cell of the animal, prior to development of the animal.

A transgenic animal of the invention can be created by introducing HICP-encoding nucleic acid into the male pronuclei of a fertilized oocyte, e.g., by microinjection, retroviral infection, and allowing the oocyte to develop in a pseudopregnant female foster animal. The HICP cDNA sequences of SEQ ID NO:1, SEQ ID NO:3 or SEQ ID NO:12 can be introduced as a transgene into the genome of a non-human animal. Intronic sequences and polyadenylation signals can also be included in the transgene to increase the efficiency of expression of the transgene. A tissue-specific regulatory sequence(s) can be operably linked to the HICP transgene to direct expression of HICP protein to particular cells. Methods for generating transgenic animals via embryo manipulation and microinjection, particularly animals such as mice, have become conventional in the art and are described, for example, in U.S. Pat. Nos. 4,736,866 and 4,870,009, both by Leder et al., U.S. Pat. No. 4,873,191 by Wagner et al. and in Hogan, B., Manipulating the Mouse Embryo, (Cold Spring Harbor Laboratory Press, Cold Spring Harbor, N.Y., 1986). Similar methods are used for production of other transgenic animals. A transgenic founder animal can be identified based upon the presence of the HICP transgene in its genome and/or expression of HICP mRNA in tissues or cells of the animals. A transgenic founder animal can then be used to breed additional animals carrying the transgene. Moreover, transgenic animals carrying a transgene encoding HICP can further be bred to other transgenic animals carrying other transgenes.

To create a homologous recombinant animal, a vector is prepared which contains at least a portion of a HICP gene into which a deletion, addition or substitution has been introduced to thereby alter, e.g., functionally disrupt, the HICP gene. The HICP gene can be a human gene, but more preferably, is a non-human homologue (for e.g., SEQ ID NO:1 SEQ ID NO:3 or SEQ ID NO:12) of a human HICP gene. For example, a rat HICP gene can be used to construct a homologous recombination vector suitable for altering an endogenous HICP gene in the rat genome. In a preferred embodiment, the vector is designed such that, upon homologous recombination, the endogenous HICP gene is functionally disrupted (i.e., no longer encodes a functional protein; also referred to as a “knock out” vector). Alternatively, the vector can be designed such that, upon homologous recombination, the endogenous HICP gene is mutated or otherwise altered but still encodes functional protein (e.g., the upstream regulatory region can be altered to thereby alter the expression of the endogenous HICP protein). In the homologous recombination vector, the altered portion of the HICP gene is flanked at its 5′ and 3′ ends by additional nucleic acid of the HICP gene to allow for homologous recombination to occur between the exogenous HICP gene carried by the vector and an endogenous HICP gene in an embryonic stem cell. The additional flanking HICP nucleic acid is of sufficient length for successful homologous recombination with the endogenous gene. Typically, several kilobases of flanking DNA (both at the 5′ and 3′ ends) are included in the vector (see e.g., Thomas, K. R. and Capecchi, M. R. (1987) Cell 51:503 for a description of homologous recombination vectors). The vector is introduced into an embryonic stem cell line (e.g., by electroporation) and cells in which the introduced HICP gene has homologously recombined with the endogenous HICP gene are selected (see e.g., Li, E. et al. (1992) Cell 69:915). The selected cells are then injected into a blastocyst of an animal (e.g., a mouse) to form aggregation chimeras (see e.g., Bradley, A. in Teratocarcinomas and Embryonic Stem Cells: A Practical Approach, E. J. Robertson, ed. (IRL, Oxford, 1987) pp. 113-152). A chimeric embryo can then be implanted into a suitable pseudopregnant female foster animal and the embryo brought to term. Progeny harboring the homologously recombined DNA in their germ cells can be used to breed animals in which all cells of the animal contain the homologously recombined DNA by germline transmission of the transgene. Methods for constructing homologous recombination vectors and homologous recombinant animals are described further in Bradley, A. (1991) Current Opinion in Biotechnology 2:823-829 and in PCT International Publication Nos.: WO 90/11 354 by Le Mouellec et al.; WO 91/01140 by Smithies et al.; WO 92/0968 by Zijlstra et al.; and WO 93/04169 by Berns et al.

In another embodiment, transgenic non-humans animals can be produced which contain selected systems which allow for regulated expression of the transgene. One example of such a system is the cre/loxP recombinase system of bacteriophage P1. For a description of the cre/loxP recombinase system, see, e.g., Lakso et al. (1992) PNAS 89:6232-6236. Another example of a recombinase system is the FLP recombinase system of Saccharomyces cerevisiae (O'Gorman et al. (1991) Science 251:1351-1355. If a cre/loxP recombinase system is used to regulate expression of the transgene, animals containing transgenes encoding both the Cre recombinase and a selected protein are required. Such animals can be provided through the construction of “double” transgenic animals, e.g., by mating two transgenic animals, one containing a transgene encoding a selected protein and the other containing a transgene encoding a recombinase.

Clones of the non-human transgenic animals described herein can also be produced according to the methods described in Wilmut, I. et al. (1997) Nature 385:810-813. In brief, a cell, e.g., a somatic cell, from the transgenic animal can be isolated and induced to exit the growth cycle and enter Go phase. The quiescent cell can then be fused, e.g., through the use of electrical pulses, to an enucleated oocyte from an animal of the same species from which the quiescent cell is isolated. The recontructed oocyte is then cultured such that it develops to morula or blastocyte and then transferred to pseudopregnant female foster animal. The offspring borne of this female foster animal will be a clone of the animal from which the cell, e.g., the somatic cell, is isolated.

IV. Pharmaceutical Compositions

The HICP nucleic acid molecules, HICPpolypeptides (particularly fragments of HICP), HICP modulators, and anti-HICP antibodies (also referred to herein as “active compounds”) of the invention can be incorporated into pharmaceutical compositions suitable for administration to a subject, e.g., a human. Such compositions typically comprise the nucleic acid molecule, polypeptide, modulator, or antibody and a pharmaceutically acceptable carrier. As used herein the language “pharmaceutically acceptable carrier” is intended to include any and all solvents, dispersion media, coatings, antibacterial and antifungal agents, isotonic and absorption delaying agents, and the like, compatible with pharmaceutical administration. The use of such media and agents for pharmaceutically active substances is well known in the art. Except insofar as any conventional media or agent is incompatible with the active compound, such media can be used in the compositions of the invention. Supplementary active compounds can also be incorporated into the compositions.

A pharmaceutical composition of the invention is formulated to be compatible with its intended route of administration. Examples of routes of administration include parenteral, e.g., intravenous, intradermal, subcutaneous, oral (e.g., inhalation), transdermal (topical), transmucosal, and rectal administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.) or phosphate buffered saline (PBS). In all cases, the composition must be sterile and should be fluid to the extent that easy syringability exists. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyetheylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as manitol, sorbitol, sodium chloride in the composition. Prolonged absorption of the injectable compositions can be brought about by including in the composition an agent which delays absorption, for example, aluminum monostearate and gelatin.

Sterile injectable solutions can be prepared by incorporating the active compound (e.g., an FLH2882 polypeptide or anti-FLH2882 antibody) in the required amount in an appropriate solvent with one or a combination of ingredients enumerated above, as required, followed by filtered sterilization. Generally, dispersions are prepared by incorporating the active compound into a sterile vehicle which contains a basic dispersion medium and the required other ingredients from those enumerated above. In the case of sterile powders for the preparation of sterile injectable solutions, the preferred methods of preparation are vacuum drying and freeze-drying which yields a powder of the active ingredient plus any additional desired ingredient from a previously sterile-filtered solution thereof.

Oral compositions generally include an inert diluent or an edible carrier. They can be enclosed in gelatin capsules or compressed into tablets. For the purpose of oral therapeutic administration, the active compound can be incorporated with excipients and used in the form of tablets, troches, or capsules. Oral compositions can also be prepared using a fluid carrier for use as a mouthwash, wherein the compound in the fluid carrier is applied orally and swished and expectorated or swallowed. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

For administration by inhalation, the compounds are delivered in the form of an aerosol spray from pressured container or dispenser which contains a suitable propellant, e.g., a gas such as carbon dioxide, or a nebulizer.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art.

The compounds can also be prepared in the form of suppositories (e.g., with conventional suppository bases such as cocoa butter and other glycerides) or retention enemas for rectal delivery.

In one embodiment, the active compounds are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811.

It is especially advantageous to formulate oral or parenteral compositions in dosage unit form for ease of administration and uniformity of dosage. Dosage unit form as used herein refers to physically discrete units suited as unitary dosages for the subject to be treated; each unit containing a predetermined quantity of active compound calculated to produce the desired therapeutic effect in association with the required pharmaceutical carrier. The specification for the dosage unit forms of the invention are dictated by and directly dependent on the unique characteristics of the active compound and the particular therapeutic effect to be achieved, and the limitations inherent in the art of compounding such an active compound for the treatment of individuals.

The nucleic acid molecules of the invention can be inserted into vectors and used as gene therapy vectors. Gene therapy vectors can be delivered to a subject by, for example, intravenous injection, local administration (see U.S. Pat. No. 5,328,470) or by stereotactic injection (see e.g., Chen et al. (1994) PNAS 91:3054-3057). The pharmaceutical preparation of the gene therapy vector can include the gene therapy vector in an acceptable diluent, or can comprise a slow release matrix in which the gene delivery vehicle is imbedded. Alternatively, where the complete gene delivery vector can be produced intact from recombinant cells, e.g. retroviral vectors, the pharmaceutical preparation can include one or more cells which produce the gene delivery system.

The pharmaceutical compositions can be included in a container, pack, or dispenser together with instructions for administration.

V. Uses and Methods of the Invention

The nucleic acid molecules, proteins, protein homologues, modulators, and antibodies described herein can be used in one or more of the following methods: 1) drug screening assays; 2) diagnostic assays; and 3) methods of treatment. A HICP protein of the invention has one or more of the activities described herein and can thus be used to, for example, modulate a heparin response in a heparin responsive cell or otherwise modulate fibroproliferation or cell proliferation. The isolated nucleic acid molecules of the invention can be used to express HICP protein (e.g., via a recombinant expression vector in a host cell in gene therapy applications), to detect HICP mRNA (e.g., in a biological sample) or a genetic lesion in an HICP gene, and to modulate HICP activity, as described further below. In addition, the HICP proteins can be used to screen drugs or compounds which modulate HICP protein activity or the HICP proteins or polypeptides can be used to screen for a specific heparin species which has an anti-proliferative activity. Moreover, HICP proteins and nucleic acid molecules encoding a HICP protein can be used as a HICP agent to treat disorders characterized by aberrant or abnormal cell proliferation, insufficient production of HICP protein or production of HICP protein forms which have decreased activity compared to wild type HICP. Moreover, the anti-HICP antibodies of the invention can be used to detect and isolate HICP protein and modulate HICP protein activity.

a. Drug Screening Assays:

The invention provides methods for identifying compounds or agents which can be used to treat disorders characterized by (or associated with) abnormal HICP nucleic acid expression and/or HICP protein activity. These methods are also referred to herein as drug screening assays and typically include the step of screening a candidate/test compound or agent for the ability to interact with (e.g., bind to) a HICP protein, to modulate the interaction of a HICP protein and a target molecule, and/or to modulate HICP nucleic acid expression and/or HICP protein activity. Candidate/test compounds or agents which have one or more of these abilities can be used as drugs to treat disorders characterized by abnormal HICP nucleic acid expression and/or HICP protein activity. Candidate/test compounds such as small molecules, e.g., small organic molecules, and other drug candidates can be obtained, for example, from combinatorial and natural product libraries.

In one embodiment, the invention provides screening assays to identify candidate/test compounds which modulate (e.g., stimulate or inhibit) the interaction (and most likely HICP activity as well) between an HICP protein and a molecule (target molecule) with which the HICP protein normally interacts. Examples of such target molecules includes proteins in the same signaling path as the HICP protein, e.g., proteins which may function upstream (including both stimulators and inhibitors of activity) or downstream of the HICP protein in a signaling pathway e.g., a heparin-induced signaling pathway. Typically, the assays are cell-free assays which include the steps of combining a HICP protein or a biologically active portion thereof, a HICP target molecule (e.g., a HICP ligand) and a candidate/test compound, e.g., under conditions wherein but for the presence of the candidate compound, the HICP protein or biologically active portion thereof interacts with (e.g., binds to) the target molecule, and detecting the formation of a complex which includes the HICP protein and the target molecule or detecting the interaction/reaction of the HICP protein and the target molecule. Detection of complex formation can include direct quantitation of the complex by, for example, measuring inductive effects of the HICP protein. A statistically significant change, such as a decrease, in the interaction of the HICP and target molecule (e.g., in the formation of a complex between the HICP and the target molecule) in the presence of a candidate compound (relative to what is detected in the absence of the candidate compound) is indicative of a modulation (e.g., stimulation or inhibition) of the interaction between the HICP protein and the target molecule. Modulation of the formation of complexes between the HICP protein and the target molecule can be quantitated using, for example, an immunoassay.

To perform the above drug screening assays, it is desirable to immobilize either HICP or its target molecule to facilitate separation of complexes from uncomplexed forms of one or both of the proteins, as well as to accommodate automation of the assay. Interaction (e.g., binding of) of HICP to a target molecule, in the presence and absence of a candidate compound, can be accomplished in any vessel suitable for containing the reactants. Examples of such vessels include microtitre plates, test tubes, and micro-centrifuge tubes. In one embodiment, a fusion protein can be provided which adds a domain that allows the protein to be bound to a matrix. For example, glutathione-S-transferase/HICP fusion proteins can be adsorbed onto glutathione sepharose beads (Sigma Chemical, St. Louis, Mo.) or glutathione derivatized microtitre plates, which are then combined with the cell lysates (e.g. ³⁵S-labeled) and the candidate compound, and the mixture incubated under conditions conducive to complex formation (e.g., at physiological conditions for salt and pH). Following incubation, the beads are washed to remove any unbound label, and the matrix immobilized and radiolabel determined directly, or in the supernatant after the complexes are dissociated. Alternatively, the complexes can be dissociated from the matrix, separated by SDS-PAGE, and the level of HICP-binding protein found in the bead fraction quantitated from the gel using standard electrophoretic techniques.

Other techniques for immobilizing proteins on matrices can also be used in the drug screening assays of the invention. For example, either HICP or its target molecule can be immobilized utilizing conjugation of biotin and streptavidin. Biotinylated HICP molecules can be prepared from biotin-NHS (N-hydroxy-succinimide) using techniques well known in the art (e.g., biotinylation kit, Pierce Chemicals, Rockford, Ill.), and immobilized in the wells of streptavidin-coated 96 well plates (Pierce Chemical). Alternatively, antibodies reactive with HICP but which do not interfere with binding of the protein to its target molecule can be derivatized to the wells of the plate, and HICP trapped in the wells by antibody conjugation. As described above, preparations of a HICP-binding protein and a candidate compound are incubated in the HICP-presenting wells of the plate, and the amount of complex trapped in the well can be quantitated. Methods for detecting such complexes, in addition to those described above for the GST-immobilized complexes, include immunodetection of complexes using antibodies reactive with the HICP target molecule, or which are reactive with HICP protein and compete with the target molecule; as well as enzyme-linked assays which rely on detecting an enzymatic activity associated with the target molecule.

In yet another embodiment, the invention provides a method for identifying a compound (e.g., a screening assay) capable of use in the treatment of a disorder characterized by (or associated with) aberrant cell proliferation and/or abnormal HICP nucleic acid expression or HICP protein activity. This method typically includes the step of assaying the ability of the compound or agent to modulate the expression of the HICP nucleic acid or the activity of the HICP protein thereby identifying a compound for treating a disorder characterized by aberrant cell proliferation and/or abnormal HICP nucleic acid expression or HICP protein activity. Disorders characterized by aberrant cell proliferation and/or abnormal HICP nucleic acid expression or HICP protein activity are described herein. Methods for assaying the ability of the compound or agent to modulate the expression of the HICP nucleic acid or activity of the HICP protein are typically cell-based assays. For example, cells which are sensitive to ligands, e.g., vascular smooth muscle cells, which transduce signals via a pathway involving HICP can be induced to overexpress an HICP protein in the presence and absence of a candidate compound. Candidate compounds which produce a statistically significant change in HICP-dependent responses (either stimulation or inhibition) can be identified. In one embodiment, expression of the HICP nucleic acid or activity of a HICP protein is modulated in cells and the effects of candidate compounds on the readout of interest (such as rate of cell proliferation) are measured. For example, the expression of genes which are up- or down-regulated a HICP response can be assayed. In preferred embodiments, the regulatory regions of such genes, e.g., the 5′ flanking promoter and enhancer regions, are operably linked to a detectable marker (such as luciferase) which encodes a gene product that can be readily detected. Phosphorylation of HICP or HICP target molecules can also be measured, for example, by immunoblotting.

Alternatively, modulators of HICP expression (e.g., compounds which can be used to treat a disorder characterized by aberrant cell proliferation, cell motility, extracellular matrix production and/or abnormal HICP nucleic acid expression or HICP protein activity) can be identified in a method wherein a cell is contacted with a candidate compound and the expression of HICP mRNA or protein in the cell is determined. The level of expression of HICP mRNA or protein in the presence of the candidate compound is compared to the level of expression of HICP mRNA or protein in the absence of the candidate compound. The candidate compound can then be identified as a modulator of HICP nucleic acid expression based on this comparison and be used to treat a disorder characterized by aberrant cell proliferation, cell motility, extracellular matrix production and/or HICP nucleic acid expression. For example, when expression of HICP mRNA or protein is greater (statistically significantly greater) in the presence of the candidate compound than in its absence, the candidate compound is identified as a stimulator of HICP mRNA or protein expression. Alternatively, when expression of HICP mRNA or protein is less (statistically significantly less) in the presence of the candidate compound than in its absence, the candidate compound is identified as an inhibitor of HICP mRNA or protein expression. The level of HICP mRNA or protein expression in the cells can be determined by methods described herein for detecting HICP mRNA or protein.

In yet another aspect of the invention, the HICP proteins can be used as “bait proteins” in a two-hybrid assay (see, e.g., U.S. Pat. No. 5,283,317; Zervos et al. (1993) Cell 72:223-232; Madura et al. (1993) J. Biol. Chem. 268:12046-12054; Bartel et al. (1993) Biotechniques 14:920-924; Iwabuchi et al. (1993) Oncogene 8:1693-1696; and Brent WO94/10300), to identify other proteins, which bind to or interact with HICP (“HICP-binding proteins” or “HICP-bp”) and modulate HICP protein activity. Such HICP-binding proteins are also likely to be involved in signal pathways with the HICP proteins as, for example, upstream or downstream elements of the heparin signal pathway.

The two-hybrid system is based on the modular nature of most transcription factors, which consist of separable DNA-binding and activation domains. Briefly, the assay utilizes two different DNA constructs. In one construct, the gene that codes for HICP is fused to a gene encoding the DNA binding domain of a known transcription factor (e.g., GAL-4). In the other construct, a DNA sequence, from a library of DNA sequences, that encodes an unidentified protein (“prey” or “sample”) is fused to a gene that codes for the activation domain of the known transcription factor. If the “bait” and the “prey” proteins are able to interact, in vivo, forming an HICP-dependent complex, the DNA-binding and activation domains of the transcription factor are brought into close proximity. This proximity allows transcription of a reporter gene (e.g., LacZ) which is operably linked to a transcriptional regulatory site responsive to the transcription factor. Expression of the reporter gene can be detected and cell colonies containing the functional transcription factor can be isolated and used to obtain the cloned gene which encodes the protein which interacts with HICP.

Modulators of HICP protein activity and/or HICP nucleic acid expression identified according to these drug screening assays can be to treat, for example, cardiovascular diseases or disorders such as ischemia/reperfusion, hypertension, and restenosis. Examples of other cardiovascular diseases or disorders which can be treated using modulators of HICP protein activity and/or nucleic acid expression are described in Robbins, S. L. et al. eds. Pathologic Basis of Disease (W.B. Saunders Company, Philadelphia, Pa. 1984) 502-547. These methods of treatment include the steps of administering the modulators of HICP protein activity and/or nucleic acid expression, e.g., in a pharmaceutical composition as described in subsection IV above, to a subject in need of such treatment, e.g., a subject with cardiovascular disease. In addition, modulators of HICP protein activity and/or HICP nucleic acid expression identified according to these drug screening assays can also be to treat proliferative disorders, for example, cell proliferation or fibroproliferative disorders such as post-surgical scarring, trauma and acute fibrosis, fibrosis of major organs and other fibroproliferative disorders.

This invention further pertains to novel HICP agents such as HICP proteins or biologically active portions thereof, HICP variants which function as antiproliferative agents and nucleic acid molecules encoding a HICP protein or variant, which can be screened to determine the efficacy of such agents on cellular proliferation (e.g., the efficacy on growth factor stimulated cellular proliferation as well as the efficacy on heparin-induced cellular proliferation).

In one embodiment, determining the ability of a HICP agent to modulate cell proliferation or fibroproliferation can be accomplished by testing the ability of HICP to interfere with the mitogenic or chemotactic activity of a growth factor, e.g., CTGF. In addition, the ability of a HICP agent to modulate cell proliferation can be accomplished by determining the ability of cells to proliferate in the absence and presence of HICP protein expression.

It is also within the scope of this invention to further use a HICP agent as described herein in an appropriate animal model. For example, an agent as described herein (e.g., a cell proliferation modulating agent) can be used in an animal model to determine the efficacy, toxicity, or side effects of treatment with such an agent. Alternatively, a HICP agent identified as described herein can be used in an animal model to determine the mechanism of action of such an agent. Animal models which are recognized in the art as predictive of results in humans with various hyperproliferation disorders are known in the art. Furthermore, this invention pertains to uses of HICP agents and agents identified by the above-described screening assays for treatments as described herein.

The invention further pertains to novel HICP proteins or polypeptides which function as affinity or screening reagents for isolating species of heparin which retain antiproliferative activity but have reduced levels of other biological activities of heparin such as anticoagulant activity. Heparin is a sub-class of the heparin sulfate class of complex carbohydrates called glycoaminoglycans. Glycoaminoglycans are composed of repeating disaccharide which can have considerable chemical variety within the oligosacchride chains in the particular gylcoaminoglycan class. Thus, in a typical composition, e.g., solution, of clinical grade heparin, over a hundred unique oligonucleotide chains would be found, all of which meet the basic chemical definition for heparin. Moreover, variety among oligosacchride chains in different heparin species imparts different biological activities or levels of biological activity to heparin species (e.g., anticoagulant activity or antiproliferative activity). The biological activities of heparin include inhibiting cell proliferation (i.e., antiproliferative activity) and modulating several enzymes involved in the anticoagulation cascade (i.e., anticoagulant activity). In addition, one of the multiple biological activities of heparin is that it has the negative side effect of reducing platelets levels in blood, thereby causing thrombocytopenia. Thus, it is desirable to separate different species of heparin in order to isolate species of heparin or fragments of heparin which have antiproliferative activity but exhibit reduced levels of other heparin biological activities as compared to other heparin species. It is, therefore, within the scope of the invention to use HICP proteins or polypeptides as affinity reagents or screening reagents to isolate heparin species which have antiproliferative activity.

In one embodiment, an affinity column of HICP protein can be used to isolate specific heparin species. Methods of preparing affinity columns are known in the art and described, for example, in Lam et al. (1976) Biochem. Biophys. Res. Commun. 69:570-577, and Hook et al. (1976) FEBS Letters 66:90-93. In another embodiment, any of the screening assays, as described above, can employ a HICP protein or polypeptide to screen for specific heparin species.

The HICP affinity reagent or screening reagent can be the entire HICP protein (e.g., the amino acid sequence of SEQ ID NO:2) or a portion thereof such as the mature HICP protein (e.g., the amino acid sequence of SEQ ID NO:13). Alternatively, the TSP1 domain (e.g., the amino acid sequence of SEQ ID NO:11) can be used as the HICP affinity reagent or screening reagent. Moreover, the HICP affinity reagent or screening reagent can comprise the TSP 1 motif (e.g., the amino acid sequence of SEQ ID NO:7) of the TSP1 domain which contains a heparin binding sequence.

In another embodiment, the invention features a method of isolating a heparin species which has antiproliferative activity. The method comprises contacting a HICP affinity reagent with a composition, e.g., a solution, containing several heparin species and isolating the heparin species which binds to the HICP affinity reagent to thereby obtain a heparin species with antiproliferative activity. This method is useful for obtaining species of heparin which have antiproliferative activity but exhibit reduced levels of other heparin biological activities as compared to other species of heparin.

b. Diagnostic Assays:

The invention further provides a method for detecting the presence of HICP in a biological sample. The method involves contacting the biological sample with a compound or an agent capable of detecting HICP protein or mRNA such that the presence of HICP is detected in the biological sample. A preferred agent for detecting HICP mRNA is a labeled or labelable nucleic acid probe capable of hybridizing to HICP mRNA. The nucleic acid probe can be, for example, the full-length HICP cDNA of SEQ ID NO: 1, or a portion thereof, such as an oligonucleotide of at least 15, 30, 50, 100, 250 or 500 nucleotides in length and sufficient to specifically hybridize under stringent conditions to HICP mRNA. A preferred agent for detecting HICP protein is a labeled or labelable antibody capable of binding to HICP protein. Antibodies can be polyclonal, or more preferably, monoclonal. An intact antibody, or a fragment thereof (e.g., Fab or F(ab′)₂) can be used. The term “labeled or labelable”, with regard to the probe or antibody, is intended to encompass direct labeling of the probe or antibody by coupling (i.e., physically linking) a detectable substance to the probe or antibody, as well as indirect labeling of the probe or antibody by reactivity with another reagent that is directly labeled. Examples of indirect labeling include detection of a primary antibody using a fluorescently labeled secondary antibody and end-labeling of a DNA probe with biotin such that it can be detected with fluorescently labeled streptavidin. The term “biological sample” is intended to include tissues, cells and biological fluids isolated from a subject, as well as tissues, cells and fluids present within a subject. That is, the detection method of the invention can be used to detect HICP mRNA or protein in a biological sample in vitro as well as in vivo. For example, in vitro techniques for detection of HICP mRNA include Northern hybridizations and in situ hybridizations. In vitro techniques for detection of HICP protein include enzyme linked immunosorbent assays (ELISAs), Western blots, immunoprecipitations and immunofluorescence. Alternatively, HICP protein can be detected in vivo in a subject by introducing into the subject a labeled anti-HICP antibody. For example, the antibody can be labeled with a radioactive marker whose presence and location in a subject can be detected by standard imaging techniques.

In one preferred embodiment of the detection method, the biological sample is a endothelial cell sample. The endothelial cell sample can comprise vascular tissue or a suspension of endothelial cells. A tissue section, for example, a freeze-dried or fresh frozen section of vascular tissue removed from a patient, can be used as the endothelial cell sample. Alternatively, the biological sample can comprise a biological fluid obtained from a subject having a cardiovascular disorder or other proliferative disorder.

The invention also encompasses kits for detecting the presence of HICP in a biological sample. For example, the kit can comprise a labeled or labelable compound or agent capable of detecting HICP protein or mRNA in a biological sample; means for determining the amount of HICP in the sample; and means for comparing the amount of HICP in the sample with a standard. The compound or agent can be packaged in a suitable container. The kit can further comprise instructions for using the kit to detect HICP mRNA or protein.

The methods of the invention can also be used to detect genetic lesions in a HICP gene, thereby determining if a subject with the lesioned gene is at risk for a disorder characterized by aberrant or abnormal HICP nucleic acid expression or HICP protein activity as defined herein. In preferred embodiments, the methods include detecting, in a sample of cells from the subject, the presence or absence of a genetic lesion characterized by at least one of an alteration affecting the integrity of a gene encoding a HICP protein, or the misexpression of the HICP gene. For example, such genetic lesions can be detected by ascertaining the existence of at least one of 1) a deletion of one or more nucleotides from a HICP gene; 2) an addition of one or more nucleotides to a HICP gene; 3) a substitution of one or more nucleotides of a HICP gene, 4) a chromosomal rearrangement of a HICP gene; 5) an alteration in the level of a messenger RNA transcript of a HICP gene, 6) aberrant modification of a HICP gene, such as of the methylation pattern of the genomic DNA, 7) the presence of a non-wild type splicing pattern of a messenger RNA transcript of a HICP gene, 8) a non-wild type level of a HICP-protein, 9) allelic loss of a HICP gene, and 10) inappropriate post-translational modification of a HICP-protein. As described herein, there are a large number of assay techniques known in the art which can be used for detecting lesions in a HICP gene.

In certain embodiments, detection of the lesion involves the use of a probe/primer in a polymerase chain reaction (PCR) (see, e.g. U.S. Pat. Nos. 4,683,195 and 4,683,202), such as anchor PCR or RACE PCR, or, alternatively, in a ligation chain reaction (LCR) (see, e.g., Landegran et al. (1988) Science 241:1077-1080; and Nakazawa et al. (1994) PNAS 91:360-364), the latter of which can be particularly useful for detecting point mutations in the HICP-gene (see Abravaya et al. (1995) Nucleic Acids Res .23:675-682). This method can include the steps of collecting a sample of cells from a patient, isolating nucleic acid (e.g., genomic, mRNA or both) from the cells of the sample, contacting the nucleic acid sample with one or more primers which specifically hybridize to a HICP gene under conditions such that hybridization and amplification of the HICP-gene (if present) occurs, and detecting the presence or absence of an amplification product, or detecting the size of the amplification product and comparing the length to a control sample.

In an alternative embodiment, mutations in a HICP gene from a sample cell can be identified by alterations in restriction enzyme cleavage patterns. For example, sample and control DNA is isolated, amplified (optionally), digested with one or more restriction endonucleases, and fragment length sizes are determined by gel electrophoresis and compared. Differences in fragment length sizes between sample and control DNA indicates mutations in the sample DNA. Moreover, the use of sequence specific ribozymes (see, for example, U.S. Pat. No. 5,498,531) can be used to score for the presence of specific mutations by development or loss of a ribozyme cleavage site.

In yet another embodiment, any of a variety of sequencing reactions known in the art can be used to directly sequence the HICP gene and detect mutations by comparing the sequence of the sample HICP with the corresponding wild-type (control) sequence. Examples of sequencing reactions include those based on techniques developed by Maxim and Gilbert ((1977) PNAS 74:560) or Sanger ((1977) PNAS 74:5463). A variety of automated sequencing procedures can be utilized when performing the diagnostic assays ((1995) Biotechniques 19:448), including sequencing by mass spectrometry (see, e.g., PCT International Publication No. WO 94/16101; Cohen et al. (1996) Adv. Chromatogr. 36:127-162; and Griffin et al. (1993) Appl. Biochem. Biotechnol. 38:147-159).

Other methods for detecting mutations in the HICP gene include methods in which protection from cleavage agents is used to detect mismatched bases in RNA/RNA or RNA/DNA duplexes (Myers et al. (1985) Science 230:1242); Cotton et al. (1988) PNAS 85:4397; Saleeba et al. (1992) Meth. Enzymol. 217:286-295), electrophoretic mobility of mutant and wild type nucleic acid is compared (Orita et al. (1989) PNAS 86:2766; Cotton (1993) Mutat Res 285:125-144; and Hayashi (1992) Genet Anal Tech Appl 9:73-79), and movement of mutant or wild-type fragments in polyacrylamide gels containing a gradient of denaturant is assayed using denaturing gradient gel electrophoresis (Myers et al (1985) Nature 313:495). Examples of other techniques for detecting point mutations include, selective oligonucleotide hybridization, selective amplification, and selective primer extension.

c. Methods of Treatment

Another aspect of the invention pertains to methods for treating a subject, e.g., a human, having a disease or disorder characterized by (or associated with) aberrant or abnormal HICP nucleic acid expression and/or HICP protein activity. These methods include the step of administering a HICP modulator to the subject such that treatment occurs. The language “aberrant or abnormal HICP expression” refers to expression of a non-wild-type HICP protein or a non-wild-type level of expression of a HICP protein. Aberrant or abnormal HICP activity refers to a non-wild-type HICP activity or a non-wild-type level of HICP activity. As the HICP protein is involved in the heparin antiproliferation mechanism, aberrant or abnormal HICP activity or expression interferes with the normal effects of heparin on heparin responsive cells. In addition, as the HICP protein can interfere with growth factor stimulated proliferation, aberrant or abnormal HICP activity or expression interferes with the normal effects of a growth factor, e.g., CTGF, on growth factor stimulated proliferation, cell motility and/or extracellular matrix production. Methods of the invention include methods for treating a subject having a disorder characterized by aberrant HICP activity or expression. These methods include administering to the subject an HICP modulator such that treatment of the subject occurs. The terms “treating” or “treatment”, as used herein, refer to reduction or alleviation of at least one adverse effect or symptom of a disease or disorder, e.g., a disease or disorder characterized by or associated with abnormal or aberrant HICP protein activity or HICP nucleic acid expression.

As used herein, a HICP modulator is a molecule which can modulate HICP nucleic acid expression and/or HICP protein activity. For example, a HICP modulator can modulate, e.g., upregulate (activate) or downregulate (suppress), HICP nucleic acid expression. In another example, a HICP modulator can modulate (e.g., stimulate or inhibit) HICP protein activity. If it is desirable to treat a disease or disorder characterized by (or associated with) aberrant or abnormal (non-wild-type) HICP nucleic acid expression and/or HICP protein activity by inhibiting HICP nucleic acid expression, a HICP modulator can be an antisense molecule, e.g., a ribozyme, as described herein. A HICP modulator which inhibits HICP nucleic acid expression can also be a small molecule or other drug, e.g., a small molecule or drug identified using the screening assays described herein, which inhibits HICP nucleic acid expression. If it is desirable to treat a disease or disorder characterized by (or associated with) aberrant or abnormal (non-wild-type) HICP nucleic acid expression and/or HICP protein activity by stimulating HICP nucleic acid expression, a HICP modulator can be, for example, a nucleic acid molecule encoding HICP (e.g., a nucleic acid molecule comprising a nucleotide sequence homologous to the nucleotide sequence of SEQ ID NO:1) or a small molecule or other drug, e.g., a small molecule (peptide) or drug identified using the screening assays described herein, which stimulates HICP nucleic acid expression.

Alternatively, if it is desirable to treat a disease or disorder characterized by (or associated with) aberrant or abnormal (non-wild-type) HICP nucleic acid expression and/or HICP protein activity by inhibiting HICP protein activity, a HICP modulator can be an anti-HICP antibody or a small molecule or other drug, e.g., a small molecule or drug identified using the screening assays described herein, which inhibits HICP protein activity. If it is desirable to treat a disease or disorder characterized by (or associated with) aberrant or abnormal (non-wild-type) HICP nucleic acid expression and/or HICP protein activity by stimulating HICP protein activity, a HICP modulator can be an active HICP protein or portion thereof (e.g., a HICP protein or portion thereof having an amino acid sequence which is homologous to the amino acid sequence of SEQ ID NO:2 or a portion thereof) or a small molecule or other drug, e.g., a small molecule or drug identified using the screening assays described herein, which stimulates HICP protein activity.

In another aspect, the invention provides a method for preventing in a subject, a disease or condition associated with aberrant cell proliferation, by administering to the subject a HICP agent which modulates cell proliferation. Subjects at risk for a disease associated with aberrant or abnormal cell proliferation can be determined by the diagnostic assays described herein or by other methods known in the art for diagnosing aberrant or abnormal cell proliferation. Administration of a HICP agent, as a prophylactic agent, can occur prior to the manifestation of symptoms characteristic of the proliferation aberrancy, such that a disease or disorder is prevented or, alternatively, delayed in its progression. Depending on the type of cell proliferation aberrancy, for example, a HICP protein or HICP variant-antagonist agent can be used for treating the subject. The appropriate HICP agent can be determined based on screening assays described herein.

Another aspect of the invention pertains to methods of modulating cell proliferation for therapeutic purposes. Non-limiting examples of disorders or diseases characterized by or associated with abnormal or aberrant cell proliferation include cardiovascular disorders and proliferative disorders (e.g., fibrotic disorders or cancers). Cardiovascular disorders are disorders which detrimentally affect normal cardiovascular function. Examples of cardiovascular disorders include atherosclerosis, ischemia/reperfusion, hypertension and restenosis. Proliferative disorders are disorders which are associated with uncontrolled or undesirable cell proliferation. Examples of proliferative disorders include fibrotic diseases or disorders (e.g., post-surgical scarring, trauma, acute fibrosis and fibrosis of major organs) as well as cancer, hyperthyroidism and psorasis.

The modulatory method of the invention involves contacting a cell with a HICP agent that modulates cell proliferation (e.g., acts as a CTGF-antagonist or directly mediates antiproliferation mechanisms). A HICP agent that modulates cell proliferation can be an agent as described herein, such as a nucleic acid encoding a HICP protein or a HICP protein, a HICP peptide, a HICP peptidomimetic. In one embodiment, the HICP agent stimulates proliferation. Examples of such stimulatory agents include an active HICP variant which serves as a HICP antagonist and a nucleic acid molecule encoding such a HICP variant that has been introduced into the cell. In another embodiment, the HICP agent inhibits cell proliferation. Examples of such inhibitory agents include HICP proteins and nucleic acid molecules encoding a HICP protein. These modulatory methods can be performed in vitro (e.g., by culturing the cell with the agent) or, alternatively, in vivo (e.g, by administering the agent to a subject). As such, the present invention provides methods of treating an individual afflicted with a disease or disorder characterized by abnormal or aberrant cell proliferation. In one embodiment, the method involves administering a HICP agent (e.g., an agent described herein), or combination of agents that modulates proliferation (e.g., upregulates or downregulates HICP expression). In another embodiment, the method involves administering a HICP protein or nucleic acid molecule as therapy to compensate for reduced HICP expression or activity or hyperproliferation.

Stimulation of cell proliferation is desirable in situations in which CTGF or other molecules involved in cell proliferation are abnormally downregulated and/or in which increased cell proliferation is likely to have a beneficial effect (e.g., impaired wound healing). Likewise, inhibition of cell proliferation is desirable in situations in which a growth factor, CTGF, is abnormally upregulated, cells are hyperproliferating and/or in which decreased cell proliferation is likely to have a beneficial effect. One example of such a situation is where a subject has a disorder characterized by aberrant cell proliferation, for example, a fibrotic disease or disorder. Another example of such a situation is where a subject has a cardiovascular disease or disorder. A HICP agent can also be used to modulate cell motility and extracellular matrix production by, for example, upregulating or downregulating HICP expression.

A subject having a cardiovascular disorder can be treated according to the present invention by administering to the subject an HICP protein or portion or a nucleic acid encoding an HICP protein or portion thereof such that treatment occurs. Similarly, a subject having a proliferative disorder can be treated according to the present invention by administering to the subject an HICP protein or portion thereof or a nucleic acid encoding an HICP protein or portion thereof such that treatment occurs.

Other aspects of the invention pertain to methods for modulating a cell associated activity. These methods include contacting the cell with an agent (or a composition which includes an effective amount of an agent) which modulates HICP activity or HICP expression such that a cell associated activity is altered relative to a cell associated activity of the cell in the absence of the agent. As used herein, “a cell associated activity” refers to a normal or abnormal activity or function of a cell. Examples of cell associated activities include proliferation, migration, differentiation, production or secretion of molecules, such as proteins, and cell survival. In a preferred embodiment, the cell is a heparin responsive cell, e.g., a cell which responds to heparin signaling through a pathway which involves HICP. An example of cells which respond to heparin signaling is vascular smooth muscle cells. In another preferred embodiment, the cell is responsive to growth factor stimulation, e.g., CTGF stimulation. An example of cells which respond to growth factor signaling, e.g., CTGF signaling, is connective tissue cells. The term “altered” as used herein refers to a change, e.g., an increase or decrease, of a cell associated activity. In one embodiment, the agent stimulates cell proliferation, cell motility and/or extracellular matrix production. In another embodiment, the agent interferes with CTGF protein activity or otherwise inhibits cell proliferation, cell motility and/or extracellular matrix production. Examples of such inhibitory agents include a HICP protein, a HICP polypeptide and a nucleotide which encodes a HICP protein. These modulatory methods can be performed in vitro (e.g., by culturing the cell with the agent) or, alternatively, in vivo (e.g., by administering the agent to a subject). In a preferred embodiment, the modulatory methods are performed in vivo, i.e., the cell is present within a subject, e.g., a mammal, e.g., a human, and the subject has a disorder or disease characterized by or associated with abnormal or aberrant cell proliferation, cell motility and/or extracellular matirx production.

A nucleic acid molecule encoding a HICP protein, a HICP protein, a HICP modulator etc. used in the methods of treatment can be incorporated into an appropriate pharmaceutical composition described herein and administered to the subject through a route which allows the molecule, protein, modulator etc. to perform its intended function. Examples of routes of administration are also described herein under subsection IV.

This invention is further illustrated by the following examples which should not be construed as limiting. The contents of all references, patent applications, patents, and published patent applications cited throughout this application are hereby incorporated by reference.

### EXAMPLES

### Example 1

### Isolation and Characterization of HICP cDNA Subtractive Hybridization Preparation of VSMC cDNA Libraries and Subtraction Libraries

In this example, the isolation of a heparin regulated gene encoding rat HICP is described.

To identify upregulated mRNA in heparin treated VSMC, a rat subtraction cDNA library which was enriched for sequences in fetal calf serum and heparin treated VSMC veruses VSCM treated only with fetal calf serum was prepared as follows: VSMC were plated at 1.2×10⁶ cells/150 mm dish and growth arrested the next day by placing them in 0.4% FCS-RPMI for 84-96 hours. Cells were released from growth arrest with 10% FCS-RPMI +/−400 μg/ml heparin, and total RNA made from the cells (Chomczynski et al. (1987) Anal. Biochem. 162:156-159) after 15 minutes, 30 minutes, 60 minutes, and 120 minutes of treatment was collected. 300 μg of total RNA from each of the four timepoints were pooled for each treatment, and poly(A)+ RNA was isolated from the two groups (pooled total RNA from FSA-treated cells and from pooled total RNA FSA and heparin-treated cells) by performing binding and elution steps from two successive oligo[dT] spin columns (Pharmacia Biotech, Piscataway, N.J.) following the manufacturer's instructions. The RNA was then used to prepare two cDNA libraries: poly(A)+ RNA from each group was primed both with oligo [dT] and random primers, and double-stranded cDNA was generated in vitro. The cDNA was ligated to adaptors and inserted in random orientation into the EcoRI site of cloning vector Lambda Zap II.

Excision of single-stranded pBluescript™ SK phage with insert from the FSA-stimulated (“S”) and the FSA+heparin-stimulated (“H”) libraries was accomplished using the procedure of Schweinfest et al. (1990) Genetic Annal of Techniques and Applications 7:64-70, except that VSC-M13 helper phage was used instead of phage R408. The DNA was purified by phenol extraction followed by dialysis. Subtracted libraries were prepared using a procedure similar to one previously described in Duguid et al. (1988) Proc. Natl. Acad. Sci 85:5738-5742. Briefly, 50 μg of single-stranded recombinant pBluescript SK from each of the cDNA libraries was sonicated then biotinylated with photobiotin (Vector Laboratories). Single-stranded recombinant pBluescript SK phage enriched for sequences expressed at higher levels in the FSA versus the FSA and heparin stimulated VSMC cells was prepared by incubation of 5 μg of S single-stranded pBluescript SK recombinant phage DNA allowing common insert sequences to hybridize. Biotinylated “H” DNA and “S” DNA hybridized to it was removed by allowing binding to avidin-agarose beads followed by centrifugation the unbiotinylated S single-stranded pBluescript SK recombinant phage bearing sequences expressed at higher levels in S than in H library remained unhybridized (not bound to biotinylated sequences) and thus remained in solution. The second strand of this subtracted DNA was synthesized in vitro, then introduced into XLI-Blue E. coli host cells (Stratagene) to make the “heparin-repressed” substraction library. The “heparin-inducted” subtraction library was made in the same way, subtracting 5 μg of H single-stranded pBluescript SK-DNA against a 10-fold excess of 10-fold excess of biotinylated, sonicated S single-stranded pBluescript SK-DNA. The transformed cells were plated on Luria both agar plates containing 50 μg/ml ampicillin and streaked with 40 μl of 0.1 M IPTG and 100 μl 2% X-Gal, and clones with possible inserts identified by blue/white screening. Plasmid DNA was made from white colonies using a DNA preparation kit (Qiagen Inc., Chatsworth, Calif.) or cesium chloride gradient method (Sambrook et al.), and the cDNA inserts recovered by digesting plasmids with EcoRI restriction enzyme, electrophoresing through agarose gels, and electroeleution of the insert DNA. The number of unique sequences isolated from the libraries was determined by cross-hybridization of dot blots of cloned DNAs to random primer-labeled insert DNAs. Slot blots and northern blots of total RNA from FSA and FSA and heparin treated VSMC prepared from cells at various times after release from growth arrest were probed with random primer-labeled insert DNAs to determine which inserts represented differentially expressed mRNAs. Of the 10 clones examined, 5 clones were expressed at higher levels in the FSA and heparin treated cells as compared to the cells treated with FSA alone.

Sequencing of cloned cDNA was carried out using Double Stranded DNA PCR Sequencing Kit (Gibco-BRL) and Sequenase™ sequencing kit.

The sequence of the isolated HICP cDNA showed homology to the cDNA sequences of several members of the CCN family (i.e., cyr61, nov, and cDNA encoding CTGF) using the BLASTX™ program. The nucleotide sequence and predicted amino acid sequence are shown in FIG. 1 (corresponding to SEQ ID NO:1 and SEQ ID NO:2, respectively). The HICP protein (corresponding to amino acids 1-250 of the predicted amino acid sequence, SEQ ID NO:2) shows 53% identity to both human CTGF (Accession Number P29279) and murine CTGF (Accession Number P29268). The percent identity was calculated using the alignment generated using MegAlign™ sequence alignment software. The initial pairwise alignment step was performed using a Wilbur-Lipmann algorithm with a K-tuple of 2, a GAP penalty of 5, a window of 4, and diagonals saved set to =4. The multiple alignment step was performed using the Clustal algorithm with a PAM 250 residue weight Table, a GAP penalty of 10, and a GAP length penalty of 10.

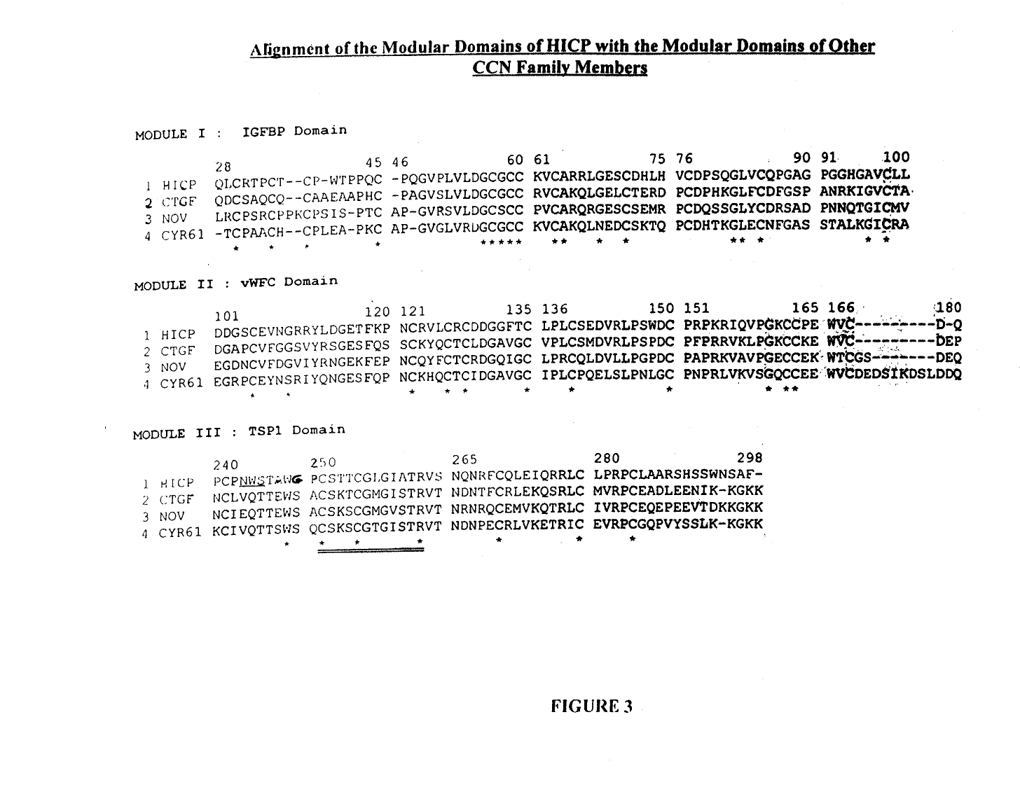

This rat HICP protein contains an IGFBP domain (corresponding to amino acids 24-93 of the predicted amino acid sequence, SEQ ID NO:2) with an IGFBP motif (corresponding to amino acids 49-56 of the predicted amino acid sequence, SEQ ID NO:2), a VWC domain (corresponding to amino acids 96-165 of SEQ ID NO:2) and a TSP1domain (corresponding to amino acids 193-250 of the predicted amino acid sequence, SEQ ID NO:2) with a TSP1 motif (corresponding to amino acids 201-215 of SEQ ID NO:2). These three modular domains of rat HICP also show significant identity to the corresponding domains in CTGF, Nov and Cyr6l as demonstrated in FIG. 3. The alignment step was performed using the Clustal algorithm as described above. In addition, the rat HICP protein contains a signal sequence (corresponding to amino acids 1-23 of the predicted amino acid sequence, SEQ ID NO:2). The predicted molecular weight for the 250 amino acid HICP is approximately 27.5 kDa.

A BLASTN™ search of the EST database revealed the following ESTs having significant homology to the nucleotide sequence of SEQ ID NO: 1:

| | | Base Pairs | % | |
|---|---|---|---|---|
| EST Database hits | Species | Covered | Identity | Coding? |
| Accession # AA754979 | mouse | 1214-1338 | 86% | no |
| Accession # AA754979 | mouse | 884-1203 | 85% | yes |
| Accession # AA717584 | mouse | 1010-1203 | 90% | no |
| Accession # AA717584 | mouse | 889-1006 | 88% | yes |

Cell Culture

To determine if HICP expression correlates with the growth state of VSMC, HICP mRNA levels were assessed in quiescent and proliferating cells.

Rat aorta smooth muscle cells were isolated from Sprague-Dawley rats (Charles River, Wellesley, Ma.; CD strain) and characterized as previously described. The cells were identified as smooth muscle cells by their “hill and valley” growth pattern and indirect immunofluorescence straining for smooth muscle specific cc-actin. Cell cultures were maintained in 10% characterized fetal calf serum (FCS) in RPMI 1640 supplemented with penicillin-streptomycin, and glutamine. Primary cells used were not greater than passage 10. Cells were counted by Coulter Counter (Hialeah, Fla.) and 6−8×10⁵ cells were plated per 100 cm² dish and allowed to attach overnight in 10% FCS/RPMI. Cells were growth arrested by treatment in 0.4% FCS/RPMI for 72 hours. COS-7 cells were maintained in 10% FCS in DMEM. Growth inhibition assays were performed is as follows: 8×10³ cells were plated on 24 well dishes in 10% FCS/RPMI. The next day, the growth medium was removed, cells were washed once with RPMI, and were placed in 0.4% FCS/RPMI for 72 hours to induce growth arrest. Cells were then places in 10% FCS/RPMI in the presence or absence of heparin or other growth inhibitors. Cells were harvested at day 4 or 5 and cell number was counted directly using Coulter Counter. The percentage of growth inhibition was calculated as follows:

% inh.=100×[1−(net growth in heparin/net growth in 10% FCS)]

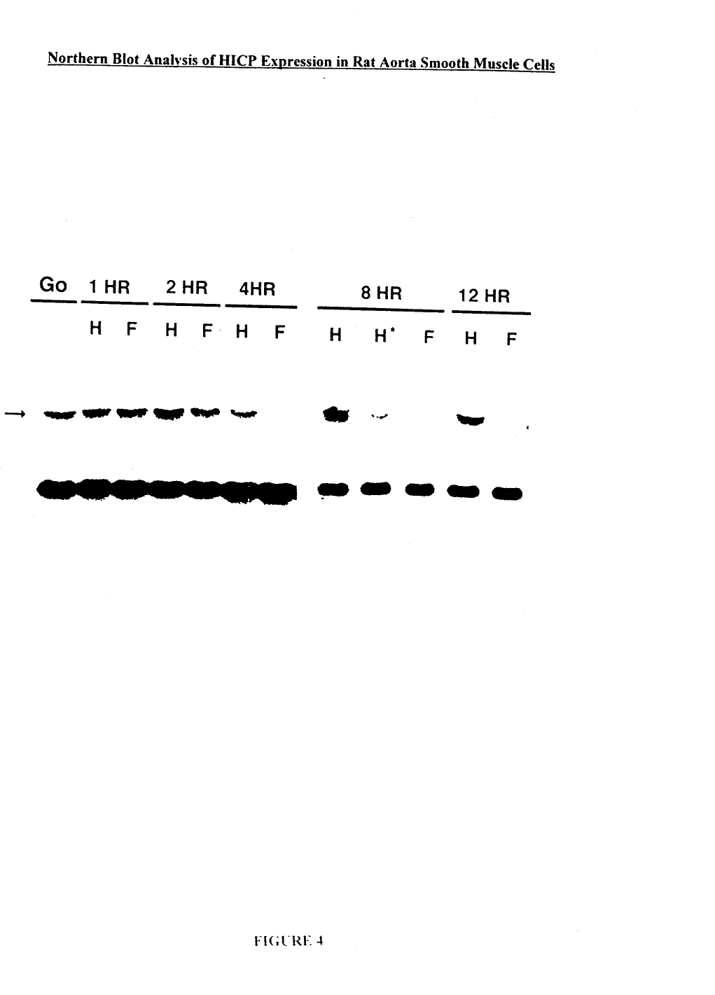

As demonstrated in FIG. 4, high levels of HICP mRNA are expressed in quiescent rat aorta smooth muscle cells (RASMC) and in RASMC exposed to FCS/RPMI containing heparin. A significant decrease in HICP mRNA expression occurs after 2 hours of exposure to FCS/RPMI alone. Thus, these results, high expression levels in quiescent cells and reduced levels in response to FCS, are consistent with the expression pattern of a growth arrest specific gene.

Moreover, heparin was demonstrated to induce HICP mRNA expression after FCS had already suppressed HICP mRNA. Delayed addition of heparin to cells treated with FCS still permitted significantly increased levels of HICP mRNA (FIG. 4, lane 9) similar to that seen when heparin and FCS were simultaneously added to quiescent cells.

HICP Expression Assays with Mitogens and Growth Inhibitors

To determine the effect of specific regulators of VSMC proliferation and identify pathways involved in the modulation of HICP mRNA expression, quiescent RASMC were treated with PDGF-BB, EGF or 10% FCS in the presence or absence of heparin or TGF-β1, IFN-β or 10% FCS in the presence or absence of heparin.

In the case of treatment of VSMC with mitogens, VSMC were isolated and cultured as described above. Growth factors were then added in the presence of 0.4% FCS/RPMI after 72 hours of exposure to 0.4% FCS/RPMI medium. Cells were counted six hours after exposure to growth factors or cells were harvested for total RNA and Northern blot analysis as described in Example 2 was performed. In addition, quiescent RASMC were treated with 10% FCS in the presence or absence of heparin (500 μg/ml), TGF-μ1 (10 μg/ml), or IFN-β (100U/ml) for four hours, at which time the RNA was harvested and Northern blot analysis was performed as described in Example 2 or the cells were counted.

Northern blot analysis revealed a reduction of HICP expression in FCS and PDGF-BB treated cells but not in EGF treated cells. The results of cell counting demonstrate that heparin inhibits the mitogenic effect of PDGF on VSMC, but does not inhibit the proliferative effect of EGF. HICP mRNA expression in TGF-β1 and IFN-β treated cells was similar to the expression pattern seen in cells treated with FCS alone. In parallel cultures, proliferation of RASMC treated with TGF-β1 and IFN-β was inhibited 45% and 77%, respectively, as determined by cell counting.

### Example 2

### Distribution of HICP mRNA in Human Tissues Northern Blot Analysis

Total RNA was extracted from heparin sensitive primary cells and cell lines and heparin resistant cell lines using 4 M guanidinium isothiocyanate and purified by centrifugation through a CsC1 cushion. Total RNA was then used for northern blot analysis. For Northern blot analysis, 10-20 μg/lane total RNA was electrophoresed on formaldehyde denaturing 1% agarose gels and transferred onto Hybond N (Amersham, Cleveland, Ohio) using the Hoefer Scientific instruments electroblot transfer apparatus (San Francisco, Calif.) and the membrane was UV cross linked. cDNA probes were radiolabeled using Random Priming Kit (Gibco-BRL) and α-³²P dATP (ICN Biomedical, Costa Mesa, Cailf.) and hybridized with membrane overnight. Blots were washed with 2× SSC and 2% SDS at 25° C. for 30 minutes and then 0.2× SSC and 0.2% SDS at 55° C. twice for 15 minutes. Blots were developed by autoradiography using Kodak-XAR or Phoshorlmager (Molecular Dynamics, Sunnyvale, Calif.). mRNA expression was quantitated by Phosphorlmager ImageQuant software.

Rat aortae were isolated from Sprague-Dawley rats (Charles River, Wellesley, Ma.; CD strain) as previously described. Aortae were stripped of any adventitia and endothelial cells were removed by hydraulic pressure via syringe leaving essentially VSMC as the only cellular constituent of the remaining tissue. Vessels were quickly frozen in liquid nitrogen, pulverized, placed in 4 M guanidinium isothiocyanate, homogenized, and centrifuged. 1.5 μg Poly A mRNA was extracted using Oligotex-dT beads from Qiagen (Chatsworth, Calif.) and electrophoresed along with RNA ladder (Gibco-BRL).

Northern blot analysis reveals HICP mRNA expression in rat aorta smooth muscle cells, as well as, high levels of expression in lung, heart, brain, and skeletal muscle. However, spleen, liver, kidneys and testes yield low or no HICP mRNA expression.

HICP Expression in Rat Aortic Endothelial Cells

To determine if heparin induces HICP mRNA expression in cells that are not responsive to the antiproliferative effect of heparin, quiescent rat aortic endothelial cells were stimulated with FCS in the presence or absence of heparin and total RNA was harvested four hours later.

Northern blot analysis showed no difference in the levels of HICP mRNA in endothelial cells treated with heparin as compared to cells treated with FCS alone. These results indicate that heparin does not up regulate HICP expression in cells unresponsive to the antiproliferative effect of heparin.

### Example 3

### Expression of Recombinant HICP Protein in Bacterial Cells

HICP can be expressed as a recombinant glutathione-S-transferase (GST) fusion polypeptide in E. coli and the fusion polypeptide can be isolated and characterized. Specifically, HICP is fused to GST and this fusion polypeptide is expressed in E. coli, e.g., strain PEB 199. As the rat HICP protein is predicted to be approximately 27.5 kDa and the GST is predicted to be approximately 26 kDa, the fusion polypeptide is predicted to be approximately 53.5 kDa in molecular weight. Expression of the GST-HICP fusion protein in PEB 199 is induced with IPTG. The recombinant fusion polypeptide is purified from crude bacterial lysates of the induced PEB 199 strain by affinity chromatography on glutathione beads. Using polyacrylamide gel electrophoretic analysis of the polypeptide purified from the bacterial lysates, the molecular weight of the resultant fusion polypeptide is determined.

### Example 4

### Expression of Recombinant HICP Protein in COS Cells

To express the HICP gene in COS cells, the pcDNA/Amp vector by Invitrogen Corporation (San Diego, Calif.) is used. This vector contains an SV40 origin of replication, an ampicillin resistance gene, an E. coli replication origin, a CMV promoter followed by a polylinker region, and an SV40 intron and polyadenylation site. A DNA fragment encoding the entire HICP protein and a HA tag (Wilson et al. (1984) Cell 37:767) fused in-frame to its 3′ end of the fragment is cloned into the polylinker region of the vector, thereby placing the expression of the recombinant protein under the control of the CMV promoter.

To construct the plasmid, the HICP DNA sequence is amplified by PCR using two primers. The 5′ primer contains the restriction site of interest followed by approximately twenty nucleotides of the HICP coding sequence starting from the initiation codon; the 3′ end sequence contains complementary sequences to the other restriction site of interest, a translation stop codon, the HA tag and the last 20 nucleotides of the HICP coding sequence. The PCR amplified fragment and the pCDNA/Amp vector are digested with the appropriate restriction enzymes and the vector is dephosphorylated using the CIAP enzyme (New England Biolabs, Beverly, Mass.). Preferably the two restriction sites chosen are different so that the HICP gene is inserted in the correct orientation. The ligation mixture is transformed into E. coli cells (strains HB101, DH5a, SURE, available from Stratagene Cloning Systems, La Jolla, Calif., can be used), the transformed culture is plated on ampicillin media plates, and resistant colonies are selected. Plasmid DNA is isolated from transformants and examined by restriction analysis for the presence of the correct fragment.

COS cells are subsequently transfected with the HICP-pcDNA/Amp plasmid DNA using the calcium phosphate or calcium chloride co-precipitation methods, DEAE-dextran-mediated transfection, lipofection, or electroporation. Other suitable methods for transfecting host cells can be found in Sambrook, J., Fritsh, E. F., and Maniatis, T. Molecular Cloning: A Laboratory Manual. 2nd, ed, Cold Spring Harbor Laboratory, Cold Spring Harbor Laboratory Press, Cold Spring Harbor, N.Y., 1989. The expression of the HICP protein is detected by radiolabelling (³⁵S-methionine or ³⁵S-cysteine available from NEN, Boston, Mass., can be used) and immunoprecipitation (Harlow, E. and Lane, D. Antibodies: A Laboratory Manual, Cold Spring Harbor Laboratory Press, Cold Spring Harbor, N.Y., 1988) using an HA specific monoclonal antibody. Briefly, the cells are labeled for 8 hours with ³⁵S-methionine (or ³⁵S-cysteine). The culture media are then collected and the cells are lysed using detergents (RIPA buffer, 150 mM NaCl, 1% NP-40, 0.1% SDS, 0.5% DOC, 50 mM Tris, pH 7.5). Both the cell lysate and the culture media are precipitated with an HA specific monoclonal antibody. Precipitated proteins are then analyzed by SDS-PAGE.

Alternatively, DNA containing the HICP coding sequence is cloned directly into the polylinker of the pCDNA/Amp vector using the appropriate restriction sites. The resulting plasmid is transfected into COS cells in the manner described above, and the expression of the HICP protein is detected by radiolabelling and immunoprecipitation using a HICP specific monoclonal antibody

### Example 5

### Culture Media Assay

To demonstrate that HICP is a secreted, soluble protein that mediates growth inhibition, culture media assays were performed using COS cells expression recombinant HICP.

Briefly, COS cells were transfected with a plasmid encoding HICP, and the cells were placed in normal growth medium for 2 days. The medium, containing products secreted by the transfected COS cells (referred to as “conditioned medium), was collected and centrifuged to remove cellular debris. Quiescent RASMC, isolated as described in Example 2, were exposed to 3H-thymidine in 10% FCS/RPMI, FCS, or to conditioned medium mixed in a 1:3 ratio with 10% FCS/RPMI. DNA synthesis was used to assess the proliferation rate of the RASMC and was measured by the amount of radiolabeled thymidine incorporated in the DNA. Cellular DNA was precipitated using trichloroacetic acid, followed by solubilization in 1 N sodium hydroxide and radioactivity was determined in counts per minute (CPM) using scintillation counting.

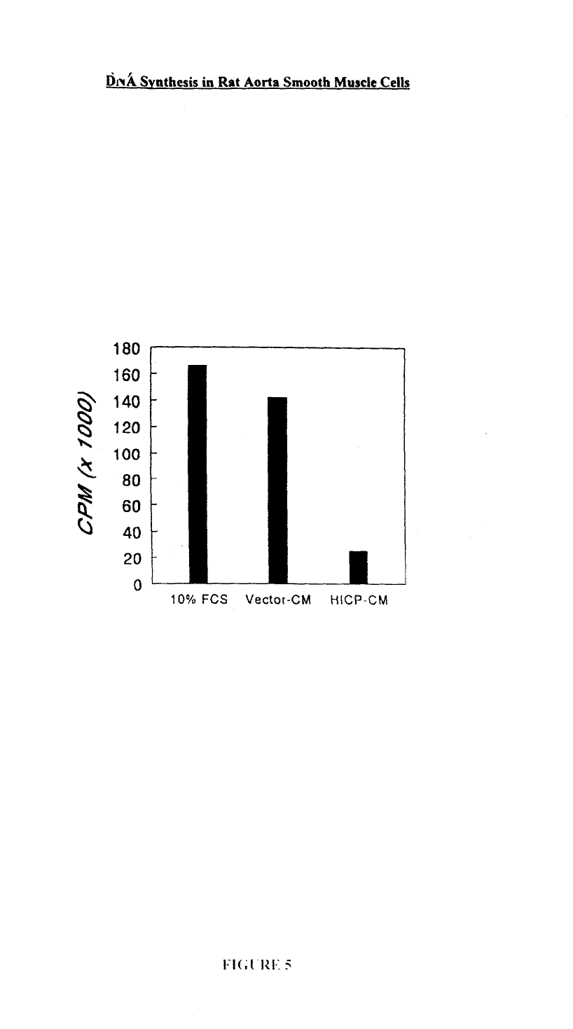

Exposure of the RASMC to the conditioned medium from COS cells transfected with HICP resulted in greater than 80% inhibition of DNA synthesis when compared with the conditioned medium of from the control COS cells transfected with vector alone. These results are demonstrated in FIG. 5.

Equivalents

Those skilled in the art will recognize, or be able to ascertain using no more than routine experimentation, many equivalents to the specific embodiments of the invention described herein. Such equivalents are intended to be encompassed by the following claims.

What is claimed is:

1. An isolated nucleic acid molecule which encodes a HICP protein, comprising a nucleotide sequence at least about 60% homologous to a nucleotide sequence of SEQ ID NO: 1, SEQ ID NO:3 or a complement thereof.

2. The isolated nucleic acid molecule of claim 1 comprising the nucleotide sequence of SEQ ID NO:1 or a complement thereof.

3. The isolated nucleic acid molecule of claim 2, further comprising nucleotides 1-883 of SEQ ID NO:1.

4. The isolated nucleic acid molecule of claim 2, further comprising nucleotides 1534-1708 of SEQ ID NO: 1.

5. The isolated nucleic acid molecule of claim 1 comprising the nucleotide sequence of SEQ ID NO:3 or a complement thereof.

6. The isolated nucleic acid molecule of claim 5, further comprising nucleotides 1-635 of SEQ ID NO:3.

7. The isolated nucleic acid molecule of claim 1 which specifically detects a HICP nucleic acid molecule relative to a nucleic acid molecule encoding a non-HICP protein.

8. An isolated nucleic acid molecule comprising a nucleotide sequence encoding a protein which comprises an amino acid sequence at least about 60% homologous to the amino acid sequence of SEQ ID NO:2.

9. The isolated nucleic acid molecule of claim 8 comprising a nucleotide sequence encoding a protein which comprises the amino acid sequence of SEQ ID NO:2.

10. An isolated nucleic acid molecule encoding a HICP protein, comprising a nucleotide sequence which hybridizes under stringent hybridization conditions to a nucleic acid molecule comprising the nucleotide sequence of SEQ ID NO:1 or SEQ ID NO:3.

11. An isolated nucleic acid molecule comprising a nucleotide sequence which hybridizes under stringent hybridization conditions to a nucleic acid molecule comprising nucleotides 1-883 of SEQ ID NO:1.

12. An isolated nucleic acid molecule at least 500 nucleotides in length which hybridizes under stringent hybridization conditions to a nucleic acid molecule comprising the nucleotide sequence of SEQ ID NO:1 or SEQ ID NO:3.

13. An isolated nucleic acid molecule which is at least about 60% homologous to a nucleotide sequence of SEQ ID NO: 1, SEQ ID NO:3 or a complement thereof, and encodes a polypeptide which has at least one of the following activities:
i) it can modulate cell proliferation;
ii) it can modulate a growth factor signaling pathway;
iii) it can modulate the activity of CTGF or PDGF; or
iv) it can modulate a heparin-induced response in a heparin-responsive cell.

14. The isolated nucleic acid molecule of claim 13 comprising the nucleotide sequence of SEQ ID NO:1 or a complement thereof.

15. The isolated nucleic acid molecule of claim 13 comprising the nucleotide sequence of SEQ ID NO:3 or a complement thereof.

16. An isolated nucleic acid molecule which is antisense to the nucleic acid molecule of any of claims 1, 9, 11 or 13.

17. A vector comprising the nucleic acid molecule of any of claims 1, 8, 10, or 13.

18. The vector of claim 17, which is a recombinant expression vector.

19. A host cell containing the vector of claim 18.

20. A method for producing HICP protein comprising culturing the host cell of claim 19 in a suitable medium until HICP protein is produced.

21. The method of claim 20, further comprising isolating HICP protein from the medium or the host cell.

22. A nonhuman transgenic animal which contains cells carrying a transgene encoding HICP protein.

23. A nonhuman homologous recombinant animal which contains cells having an altered HICP gene.

24. An isolated HICP protein comprising an amino acid sequence at least about 60% homologous to the amino acid sequence of SEQ ID NO:2.

25. An isolated HICP protein which is encoded by a nucleic acid molecule comprising a nucleotide sequence at least about 60% homologous to a nucleotide sequence of SEQ ID NO: 1, SEQ ID NO:3, or a complement thereof.

26. An isolated HICP protein which is encoded by a nucleic acid molecule comprising a nucleotide sequence which hybridizes under stringent hybridization conditions to a nucleic acid molecule comprising the nucleotide sequence of SEQ ID NO:1 or SEQ ID NO:3.

27. An isolated HICP protein which is sufficiently homolgous to the amino acid sequence of SEQ ID NO:2 and retains HICP biological activity.

28. The isolated protein of claim 27 comprising an amino acid sequence at least about 60% homologous to an amino acid sequence of SEQ ID NO:2.

29. An isolated HICP protein comprising an amino acid sequence at least about 60% homolgous to the amino acid sequence of SEQ ID NO:2 and which has at least one of the following biological activities:
i) it can modulate cell proliferation;
ii) it can modulate a growth factor signaling pathway;
iii) it can modulate the activity of CTGF or PDGF;
iv) it can modulate a heparin-induced response in a heparin-responsive cell;
v) it can modulate cell motility; or
vi) it can modulate extracellular matrix production.

30. The isolated protein of any of claims 24-29, comprising an N-terminal IGFBP motif.

31. The isolated protein of any of claims 24-29, comprising a VWC motif.

32. The isolated protein of any of claims 24-29, comprising a TSP1 motif.

33. An isolated protein comprising the amino acid sequence of SEQ ID NO:2.

34. A fusion protein comprising a HICP polypeptide operatively linked to a non-HICP polypeptide.

35. An antibody that specifically binds HICP.

36. The antibody of claim 35, which is monoclonal.

37. The antibody of claim 36, which is labeled with a detectable substance.

38. A pharmaceutical composition comprising the protein of any one of claims 24-29, or 34, and a pharmaceutically acceptable carrier.

39. A pharmaceutical composition comprising the antibody of claim 35 and a pharmaceutically acceptable carrier.

40. A method for modulating a cell-associated activity comprising contacting a cell with an agent which modulates HICP protein activity or HICP nucleic acid expression such that the cell-associated activity is altered relative to the cell-associated activity of the cell in the absence of the agent.

41. The method of claim 40, wherein the agent stimulates a HICP protein activity or expression.

42. The method of claim 40, wherein the agent inhibits a HICP protein activity or expression.

43. The method of claim 42, wherein the agent is an antisense HICP nucleic acid molecule.

44. The method of claim 42, wherein the agent is an antibody that specifically binds to HICP.

45. The method of claim 40, wherein the cell is present within a subject and the agent is administered to the subject.

46. A method for treating a subject having a disorder characterized by aberrant HICP protein activity or nucleic acid expression comprising administering to the subject a HICP modulator such that treatment of the subject occurs.

47. The method of claim 46 wherein the HICP modulator is a nucleic acid molecule encoding a HICP protein.

48. A method for detecting the presence of HICP activity in a biological sample comprising contacting a biological sample with an agent capable of detecting an indicator of HICP activity such that the presence of HICP activity is detected in the biological sample.

49. The method of claim 48, wherein the agent detects HICP mRNA.

50. The method of claim 49, wherein the agent is a labeled nucleic acid probe capable of hybridizing to HICP mRNA.

51. The method of claim 48, wherein the agent detects HICP protein.

52. The method of claim 48, wherein the agent is a labeled antibody capable of specifically binding to HICP protein.

53. A method for treating a disorder characterized by abberant cell proliferation by administering to a subject having the disorder a HICP agent which alters cell proliferation relative to cell proliferation in the absence of the agent.

54. The method of claim 53, wherein the HICP agent is a HICP protein or portion thereof.

55. The method of claim 53, wherein the HICP agent is a nulceotide encoding a HICP protein.

56. The method of claim 53, wherein the disorder is a cardiovascular disorder.

57. The method of claim 53, wherein the disorder is a fibrotic disorder.

58. A diagnostic assay for identifying a genetic alteration in a cell sample, the presence or absence of the genetic alteration characterized by at least one of (i) aberrant modification or mutation of a gene encoding a HICP protein, and (ii) mis-regulation of said gene or (iii) aberrant post-translational modification of a HICP protein.

59. The assay of claim 58, wherein detecting said alteration includes:
a. providing a reagent comprising a diagnostic probe of claim 11 or 12;
b. combining said reagent with nucleic acid of said cell sample; and
c. detecting, by hybridization of said probe to said cellular nucleic acid, the existence of at least one of a deletion of one or more nucleotides from said gene, an addition of one or more nucleotides to said gene, a substitution of one or more nucleotides of said gene, a gross chromosomal rearrangement of all or a portion of said gene, a gross alteration in the level of an mRNA transcript of said gene, or a non-wild type splicing pattern of an mRNA transcript of said gene.

60. The assay of claim 58, wherein detecting said alteration includes:
a. providing a reagent comprising two diagnostic probes;
b. combining said reagent with nucleic acid of said cell sample; and
c. detecting, by amplification or lack of amplification of said cellular nucleic acid, the absence or existence of said alteration.

61. A method for isolating a heparin specie which has antiproliferative activity comprising:
a. contacting a HICP affinity reagent with a composition containing heparin species; and
b. isolating the heparin specie which binds to the HICP affinity reagent to thereby obtain the heparin specie which has antiproliferative activity.
